(19) United States
(12) Patent Application Publication
(10) Pub. No.: US 2024/0394879 A1
(43) Pub. Date: Nov. 28, 2024

(54) EYE SEGMENTATION SYSTEM FOR TELEHEALTH MYASTHENIA GRAVIS PHYSICAL EXAMINATION

(71) Applicants: THE GEORGE WASHINGTON UNIVERSITY, Washington, DC (US); Care Constitution Corp., Newark, DE (US)

(72) Inventors: Marc P. GARBEY, Houston, TX (US); Guillaume JOERGER, Strasbourg (FR)

(73) Assignees: THE GEORGE WASHINGTON UNIVERSITY, Washington, DC (US); Care Constitution Corp., Newark, DE (US)

(21) Appl. No.: 18/791,374

(22) Filed: Jul. 31, 2024

Related U.S. Application Data
(63) Continuation of application No. PCT/US2023/032070, filed on Sep. 6, 2023.
Application No. PCT/US2023/032070 is a continuation-in-part of application No. PCT/US2023/061783, filed on Feb. 1, 2023.
(60) Provisional application No. 63/413,779, filed on Oct. 6, 2022.
(60) Provisional application No. 63/305,420, filed on Feb. 1, 2022.

(51) Int. Cl. G06T 7/00; A61B 3/113; A61B 3/14; A61B 5/00; G06T 7/11; G06T 7/246; G06V 10/25; G06V 10/70; G06V 40/16; G06V 40/18; G16H 40/67

(52) U.S. Cl. CPC G06T 7/0012; A61B 3/113; A61B 3/145; A61B 5/40; A61B 5/41; A61B 5/7465; G06T 7/11; G06T 7/246; G06V 10/25; G06V 10/70; G06V 40/171; G06V 40/18; G16H 40/67; G06T 2207/10016; G06T 2207/20081; G06T 2207/30041; G06T 2207/30201; G06V 2201/03

(57) ABSTRACT

Due to the precautions put in place during the COVID-19 pandemic, utilization of telemedicine has increased quickly for patient care and clinical trials. Unfortunately, teleconsultation is closer to a video conference than a medical consultation with the current solutions setting the patient and doctor into a discussion that relies entirely on a two-dimensional view of each other. A telehealth platform is augmented by a digital twin of the patient that assists with diagnostic testing of ocular manifestations of myasthenia gravis. A hybrid algorithm combines deep learning with computer vision to give quantitative metrics of ptosis and ocular muscle fatigue leading to eyelid droop and diplopia. The system works both on a fixed image and video in real time allowing capture of the dynamic muscular weakness during the examination. The robustness of the system can be more important that the accuracy obtained in controlled conditions, so that the system and method can operate in practical standard telehealth conditions. The approach is general and can be applied to many disorders of ocular motility and ptosis.

### CROSS-REFERENCE TO RELATED APPLICATIONS

This application claims the benefit of priority of U.S. Provisional Application Ser. No. 63/413,779 filed on Oct. 6, 2022, and PCT Application No. PCT/US2023/061783, filed Feb. 1, 2023. The content of those applications are relied upon and incorporated herein by reference in its entirety.

### GOVERNMENT LICENSE RIGHTS

This invention was made with government support under Grant No. U54 NS115054 awarded by NIH. The U.S. government has certain rights in the invention.

### BACKGROUND

Telemedicine (TM) enables practitioners and patients (including disabled patients who have difficulty traveling to in-person consultations) to interact at anytime from anywhere in the world, reducing the time and cost of transportation, reducing the risk of infection by allowing patients to receive care remotely, reducing patient wait times, and enabling practitioners to spend more of their time providing care patients. Accordingly, telemedicine has the potential to improve the efficiency of the medical consultations for patients seeking medical care, practitioners evaluating the effectiveness of a specific treatment (e.g., as part of a clinical trial), etc.

Telemedicine also provides a platform for capturing and digitizing relevant information and adding that data to the electronic health records of the patient, enabling the practitioner to for example, using voice recognition and natural language processing to assist the provider in documenting the consultation and even recognizing the patient pointing to a region of interest and selecting a keyword identifying that region of interest.

Telemedicine is also an emerging tool for monitoring patients with neuromuscular disorders and has the great potential to improve clinical care [1,2] with patients having favorable impressions to telehealth during the COVID-19 pandemic [3,4]. However, further developments and tools taking advantage of the video environment are necessary to make complete remote alternatives to physiological testing and disability assessment [2]. One such approach is provided in PCT/US23/61783, which is hereby incorporated by reference in its entirety.

Telehealth is particularly well-suited for the management of patients with myasthenia gravis (MG) due to its fluctuating severity and potential for early detection of significant exacerbations. MG is a chronic, autoimmune neuromuscular disorder, which manifests with generalized fatiguing weakness with a propensity to involve the ocular muscles. For this purpose, the Myasthenia Gravis Core Exam (MG-CE) [5] was designed to be conducted via telemedicine. The validated patient reported outcome measures typically used in clinical trials may also be added to the standard TM visit to enhance the rigor of the virtual examination [6]. The first two components of the MG-CE [5] are the evaluation of ptosis (upper eyelid droops) (Exercise 1 of the MG-CE) and diplopia (double vision) (Exercise 2 of the MG-CE).

Today's standard medical examination relies entirely on the expertise of the medical doctor who grades each Exercise of the MG-CE protocol by watching the patient. For example, the examiner rates the severity of ptosis by judging qualitatively the position of the eyelid above the pupil, and eventually noting when ptosis becomes more severe over the course of the assessment [7]. Further, the determination of diplopia is entirely dependent on the patient's report. Also, the exam is dependent on the patient's interpretation of what is meant by double vision (versus blurred vision) further complicated by the potential suppression of the false image by central adaptation, and in some situations, monocular blindness, which eliminates the complaint of double vision. The measurement of ocular motility by the present disclosure limits these challenges.

### SUMMARY

One goal of the system and method of the present disclosure is to complement the neurological exam with computer algorithms that can quantitatively and reliably report information directly to the examiner, along with some error estimate on the metric output. The algorithm should be fast enough to provide feedback in real-time, and automatically enter the medical record. A similar approach was used by Liu and colleagues, [8] monitoring patients during ocular Exercises to bring a computer-aided diagnosis, but with highly controlled data and environment. The present system takes a more versatile approach, by extracting data from more generic telehealth footage and requires as little additional effort from the patient and clinician as possible.

The present disclosure addresses the first two components of the MG-CE [5], namely the evaluation of ptosis (Exercise 1 of the MG-CE) and diplopia (Exercise 2 of the MG-CE), thus focusing on the examination of tracking eye and eyelid movement. Along these lines the algorithm works on video and captures the time dependent relaxation curves of ptosis and misalignment of both eyes that relate to fatigue. Assessing the dynamic may not be feasible by the examiner who simply watches the patient perform tasks and should leverage the value of the medical exam. It is understood that the medical doctor is the final judge of the diagnostic: the present system is a supporting tool like any AI generated image automatic annotation in radiography for example [9] and is not intended to replace the medical doctor diagnostic skill. Further, the system does not supplement the sophisticated technology used to study ocular motility for the last five decades [10].

Symptoms of double vision and ptosis are appreciated in essentially all patients with myasthenia gravis, and the evaluation of lid position and ocular motility is a key aspect of the diagnostic examination and ongoing assessment of patients. In many neurological diseases, including dementias, multiple sclerosis, strokes, and cranial nerve palsies, eye movement examination is important in diagnosis. The system and algorithm might be also useful in telehealth session targeting the diagnosis and monitoring of these neurological diseases [11,12,13]. The technology may also be utilized for assessment in the in-person setting as a means to objectively quantitate the ocular motility examination.

This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 is a diagram of a cyber-physical telehealth system, which includes a practitioner system and a patient system, according to exemplary embodiments;

FIG. 2(a) is a diagram of the patient system, which includes a patient computing system, a camera enclosure, and a hardware control box, according to exemplary embodiments;

FIG. 2(b) is a diagram of the patient system according to another exemplary embodiment;

FIG. 2(c) is a diagram of the patient system according to another exemplary embodiment

FIG. 3 is a diagram of the patient computing system of FIG. 2(a) according to exemplary embodiments;

FIG. 4(a) is a block diagram of a videoconferencing module according to exemplary embodiments;

FIG. 4(b) is a block diagram of a sensor data classification module according to exemplary embodiments;

FIG. 5(a) is a diagram of example Dlib facial landmark points;

FIG. 5(b), 5(c), 5(d) are images of example regions of interest in patient video data according to exemplary embodiments, where FIG. 5(b) shows Eye Opening distance (right eye) and eye area (shaded on left eye), and FIG. 5(d) shows Eye length measurement;

FIG. 6(a) is a block diagram of patient system controls according to exemplary embodiments;

FIG. 6(b) is a block diagram illustrating an audio calibration module, a patient tracking module, and a lighting calibration module according to exemplary embodiments;

FIG. 6(c) is a block diagram illustrating the output of visual aids to assist the patient and/or the practitioner according to exemplary embodiments;

FIG. 7 is a view of a practitioner user interface according to exemplary embodiments;

FIGS. 8(a), 8(b) show a subject looking up in Exercise 1 of the MG-CE to evaluate ptosis;

FIGS. 9(a), 9(b) show a normal subject looking eccentrically in Exercise 2 of the MG-CE to evaluate simulated Diplopia;

FIG. 10 is a graph of Blinking Identification, where each lower pick for the right and left eyes are perfectly synchronized and corresponds to blinking;

FIG. 11(a) shows local rectangle to search for the correct position of the lower lid;

FIG. 11(b) shows local rectangle to draw the interface between the iris and sclera;

FIG. 12 shows Barycentric Coordinate (a) used in Diplopia Assessment;

FIG. 13(a) shows Visual Verification on zoomed image of eyes using a 2 pixel rule with Exercise 1;

FIG. 13(b) shows Visual Verification on zoomed image of eyes using a 2 pixel rule with Exercise 2;

FIG. 14(a) is a graph of opening and holding up the right eye with a Dynamic Evaluation=[−0.15, −0.17];

FIG. 14(b) is a graph of opening and holding up the left eye with a Dynamic Evaluation=[−0.15, −0.17];

FIGS. 15(a)-15(d) are graphs that show the evolution of the barycentric coordinates of each eye during the second Exercise; a normal subject is making a convergence movement, which leads to rotation of each eye towards the midline;

FIGS. 16(a), 16(b) show example of the ptosis assessment of one of the ADAPT patient series;

FIGS. 17(a), 17(b) are flow diagrams illustrating operation of the system; and

FIG. 18 is a report generated by the system.

The figures show illustrative embodiment(s) of the present disclosure. Other embodiments can have components of different scale. Like numbers used in the figures may be used to refer to like components. However, the use of a number to refer to a component or step in a given figure has a same structure or function when used in another figure labeled with the same number, except as otherwise noted.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

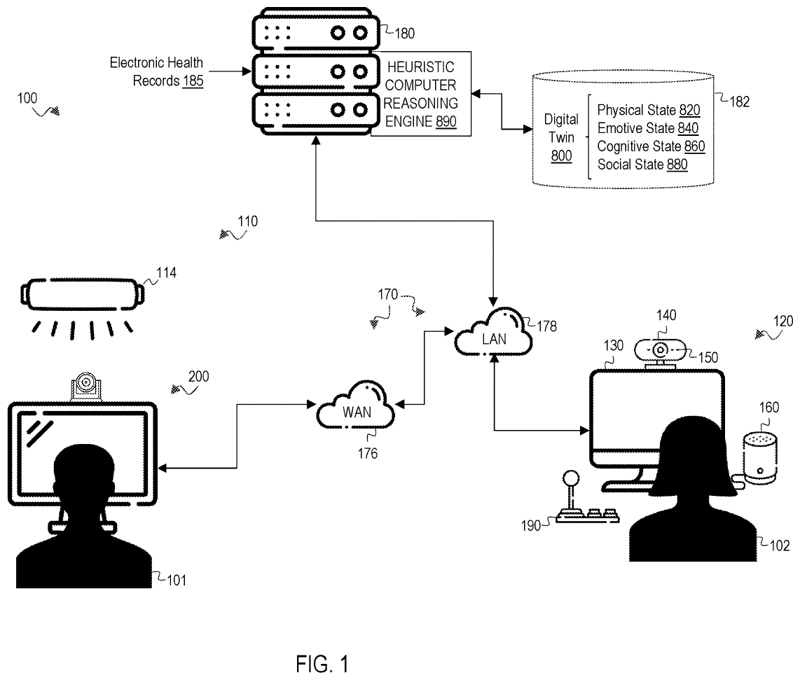

FIG. 1 is a diagram of a remotely-controllable cyber-physical telehealth system 100 according to exemplary embodiments. The telehealth system 100 can be any suitable telehealth system, such as the one shown and described in PCT/US23/61783, which is hereby incorporated by reference in its entirety.

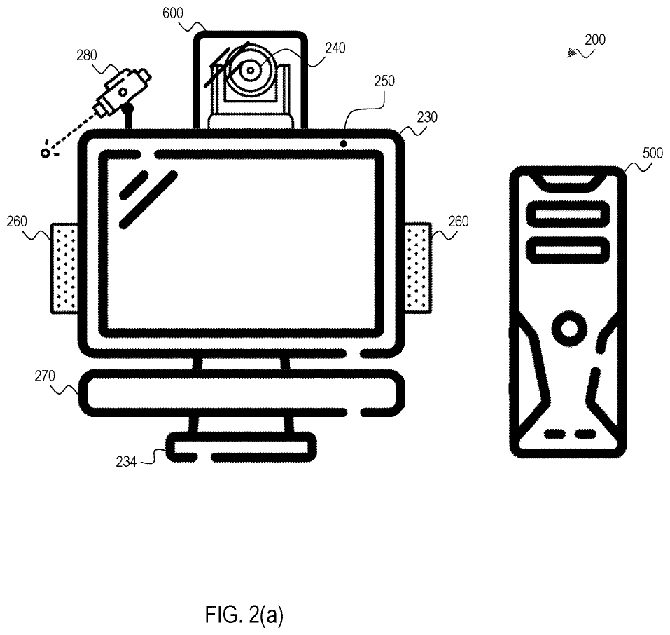

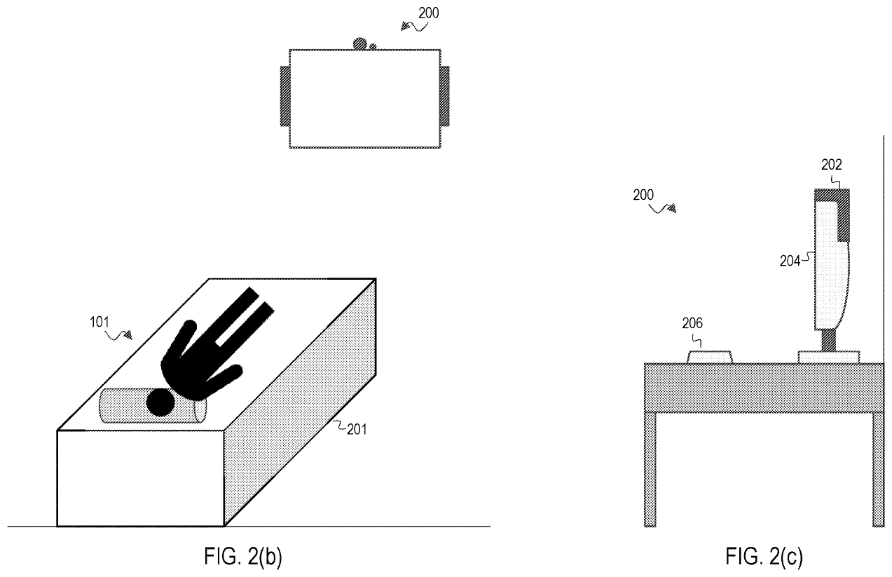

In the embodiment of FIG. 1, the cyber-physical system 100 includes a practitioner system 120 (for use by a physician or other health practitioner 102) in communication, via one or more communications networks 170, with a patient system 200 (FIG. 2(a)) and a patient computing system 500 (FIG. 2(a)) located in a patient environment 110 of a patient 101. The practitioner system 120 includes a practitioner display 130, a practitioner camera 140, a practitioner microphone 150, a practitioner speaker 160, and a patient system controller 190. In some embodiments, the patient environment 120 includes a remotely-controllable lighting system 114, which enables the brightness of the patient environment 110 to be remotely adjusted. The communications network(s) 170 may include wide area networks 176 (e.g., the Internet), local area networks 178, etc. In some embodiments, the patient computing system 500 and the practitioner system 120 are in communication with a server 180 having a database 182 to store the data from the analysis via the communications network(s) 170.

As described in detail below, the cyber-physical system 100 generates objective metrics indicative of the physical, emotive, cognitive, and/or social state of the patient 101. (Additionally, the cyber-physical system 100 may also provide functionality for the practitioner 102 to provide subjective assessments of the physical, emotive, cognitive, and/or social state of the patient 101.) Together with the electronic health records 184 of the patient 101, those objective metrics and/or subjective assessments can be used to form a digital representation of the patient 101 referred to as a digital twin 800 that includes physical state variables 820 indicative of the physical state of the patient 101, emotive state variables 840 indicative of the emotive state of the patient 101, cognitive state variables 860 indicative of the cognitive state of the patient 101, and/or social state variables 880 indicative of the social state of the patient 101. The digital twin 800, which is stored in the database 182, provides a mathematical representation of the state of the patient 101 (e.g., at each of a number of discrete points in time), which may be used by a heuristic computer reasoning engine 890 that uses artificial intelligence to support clinical diagnosis and decision-making.

FIGS. 2(a)-2(c) are diagrams of the patient system 200 according to exemplary embodiments. In the embodiment of FIG. 2(a), the patient system 200 includes a patient display 230, a patient camera 240, a thermal imaging camera 250, speakers 260, an eye tracker 270, a laser pointer 280. The patient camera 240 is a high definition, remotely-controllable pan-tilt-zoom (PTZ) camera with adjustable horizontal position (pan), vertical position (tilt), and focal length of the lens (zoom). In some embodiments, the patient display 230 may be mounted on a remotely-controllable rotating base 234, enabling the horizontal orientation of the patient display 230 to be remotely adjusted. Additionally, in some embodiments, the patient display 230 may also be mounted on a remotely-controllable vertically-adjustable mount (not shown), enabling the vertical orientation of the patient display 230 to be remotely adjusted.

As shown in FIG. 2(b), the patient system 200 may be used in clinical settings, for example by a patient 101 in a hospital bed 201. As shown in FIG. 2(c), the patient system 200 may be used in conjunction with a patient computing system 500 that includes a processing device such as, for example, a traditional desktop computer 202, for example having a display 204 and a keyboard 206. In those embodiments, for example, the patient system 200 may be realized as a compact system package that can be mounted on the display 204.

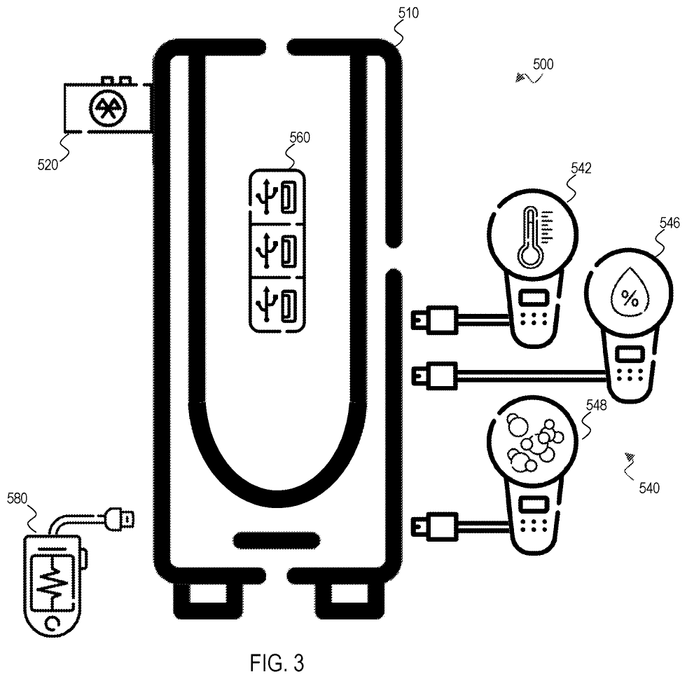

FIG. 3 is a block diagram of the patient computing system 500 according to exemplary embodiments. In the embodiment of FIG. 3, the patient computing system 500 includes a processing device such as a compact computer 510, a communications module 520, environmental sensors 540, and one or more universal serial bus (USB) ports 560. The environmental sensors 540 may include any sensor that measures information indicative of an environmental condition of the patient environment 110, such as a temperature sensor 542, a humidity sensor 546, an airborne particle senor 548, etc. In some embodiments, the patient computing system 500 may include one or more physiological sensors 580. The physiological sensors 580 may include any sensor that measures a physiological condition of the patient 101, such as a pulse oximeter, a blood pressure monitor, an electrocardiogram, etc. The physiological sensors 580 may interface with the patient computing system 500 via the USB port(s) 560, which may also provide functionality to upload physiological data from an external health monitoring device (e.g., data indicative of the sleep and/or physical activity of the patient captured by a smartwatch or other wearable activity tracking device).

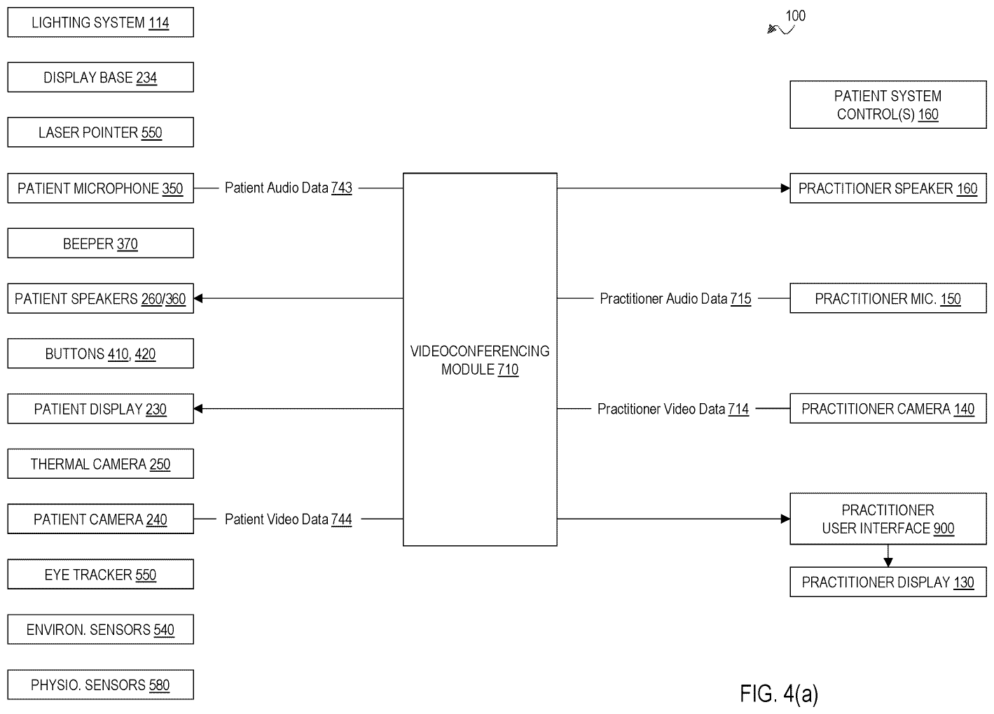

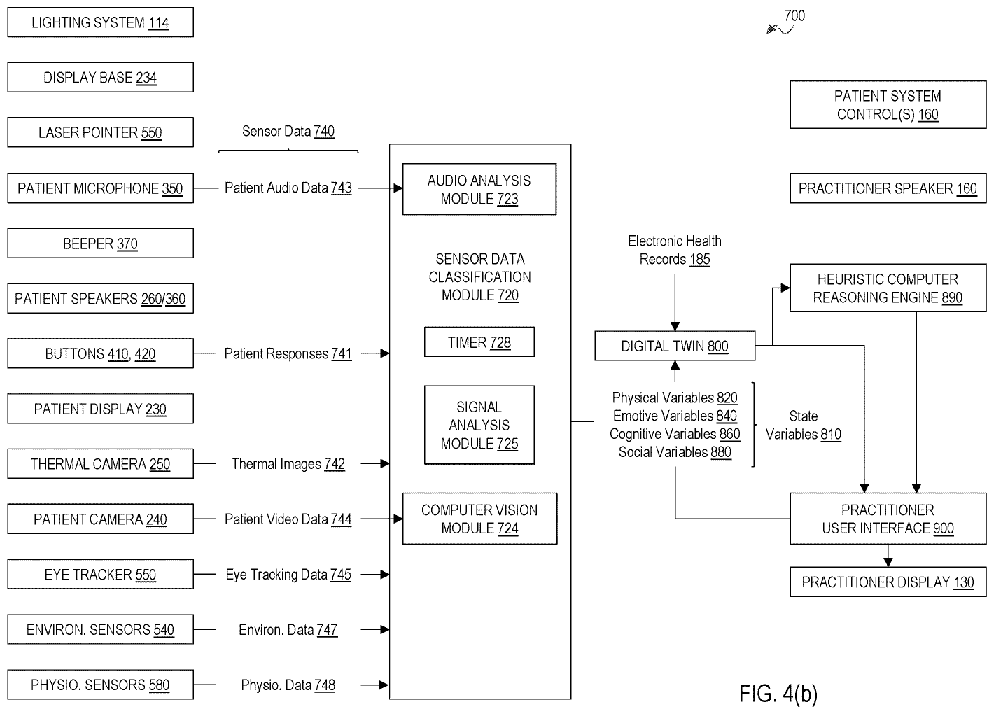

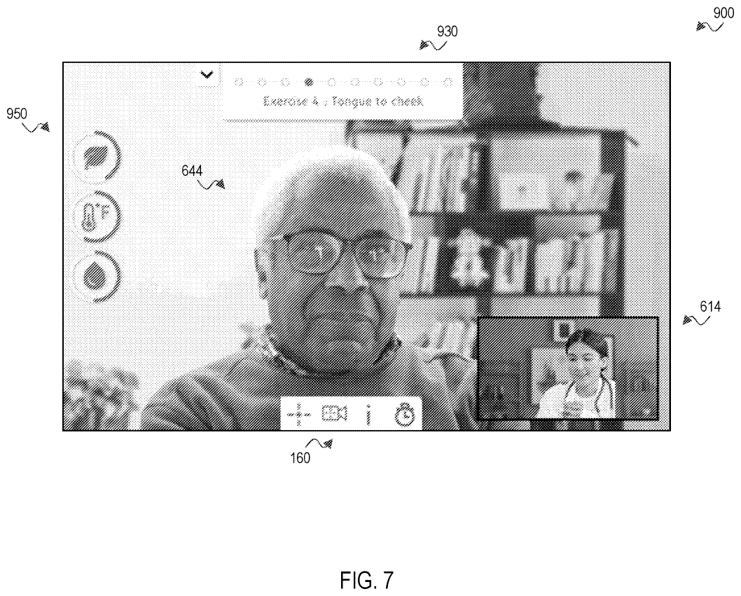

FIGS. 4(a)-6(c) are block diagrams of the software modules 700 and data flow of the cyber-physical system 100 according to exemplary embodiments. In the embodiment of FIG. 4(a), the cyber-physical system 100 includes a videoconferencing module 710, which may be realized as software instructions executed by both the patient computing system 500 and the practitioner system 120. As described above, patient audio data 743 is captured by the patient microphone 350, practitioner audio data 715 is captured by the practitioner microphone 150, practitioner video data 714 is captured by the practitioner camera 140, and patient video data 744 is captured by the patient camera 240. Similar to commercially-available videoconferencing software (e.g., Zoom), the videoconferencing module 710 outputs the patient audio data 743 via the practitioner speaker 160, outputs practitioner audio data 715 via the patient speaker(s) 260 or 360, outputs practitioner video data 714 captured by the practitioner camera 140 via the patient display 230, and outputs patient video data 744 via a practitioner user interface 900 (FIG. 7) on the practitioner display 130.

To perform the computer vision analysis described below (e.g., by the patient computing system 500), the patient video data 744 may be captured and/or analyzed at a higher resolution (and/or a higher frame rate, etc.) than is typically used for commercial video conferencing. Similarly, to perform the audio analysis described below, the patient audio data 743 may be captured and/or analyzed at a higher sampling rate, with a larger bit depth, etc., than is typical for commercial video conferencing software. Accordingly, while the patient video data 744 and the patient audio data 743 transmitted to the practitioner system 120 via the communications networks 170 may be compressed, the computer vision and audio analysis described below may be performed (e.g., by the patient computing system 500) using the uncompressed patient video data 744 and/or patient audio data 743. In other embodiments, higher resolution images and higher sampling audio rates need not be used, and standard resolution and rates can be utilized.

In the embodiment of FIG. 4(b), the cyber-physical system 100 includes a sensor data classification module 720, which includes an audio analysis module 723, a computer vision module 724, a signal analysis module 725, and a timer 728. The sensor data classification module 720 generates physical state variables 820 indicative of the physical state of the patient 101, emotive state variables 840 indicative of the emotive state of the patient 101, cognitive state variables 820 indicative of the cognitive state of the patient 101, and/or social state variables 820 indicative of the social state of the patient 101 (collectively referred to herein as state variables 810) using the patient audio data 743 is captured by the patient microphone 350, the patient video data 744 captured by the patient camera 240, patient responses 741 captured using the buttons 410 and 420, thermal images 742 captured by the thermal camera 250, eye tracking data 745 captured by the eye tracker 550, environmental data 747 captured by one or more environmental sensors 540, and/or physiological data 748 captured by one or more physiological sensors 580 (collectively referred to herein as sensor data 740).

More specifically, the sensor data classification module 720 may be configured to reduce or eliminate noise in the sensor data 740 and perform lower-level artificial intelligence algorithms to identify specific patterns in the sensor data 740 and/or classify the sensor data 740 (e.g., as belonging to one of a number of predetermined ranges). In the embodiments of FIGS. 4(b) through 6(c) described in detail below, for example, the computer vision module 724 is configured to perform computer vision analysis of the patient video data 744, the audio analysis module 723 is configured to perform audio analysis of the patient audio data 743, and the signal analysis module 725 is configured to perform classical signal analysis of the other sensor data 740 (e.g., the thermal images 742, the eye tracking data 745, the physiological data 748, and/or the environmental data 747).

The state variables 810 calculated by the sensor data classification module 720 form a digital twin 800 that may be the input of a heuristic computer reasoning engine 890. Additionally, the sensor data 740 and/or state variables 810 and recommendations from the digital twin 800 and/the heuristic reasoning engine 890 may be displayed to the practitioner 102 via the practitioner user interface 900.

In a clinical setting, for instance, the signal analysis module 725 may identify physical state variables 820 indicative of the physiological condition of the patient 101 (e.g., body temperature, pulse oxygenation, blood pressure, heart rate, etc.) based on physiological data 748 received from one or more physiological sensors 580 (e.g., a thermometer, a pulse oximeter, a blood pressure monitor, an electrocardiogram, data transferred from a wearable health monitor, etc.). Additionally, to provide functionality to identify physical state variables 820 in settings where physiological sensors 580 would be inconvenient or are unavailable, the sensor data classification module 720 may be configured to directly or indirectly identify physical state variables 820 in a non-invasive manner by performing computer vision and/or signal processing using other sensor data 740. For example, the thermal images 742 may be used to track heart beats and/or measure breathing rates.

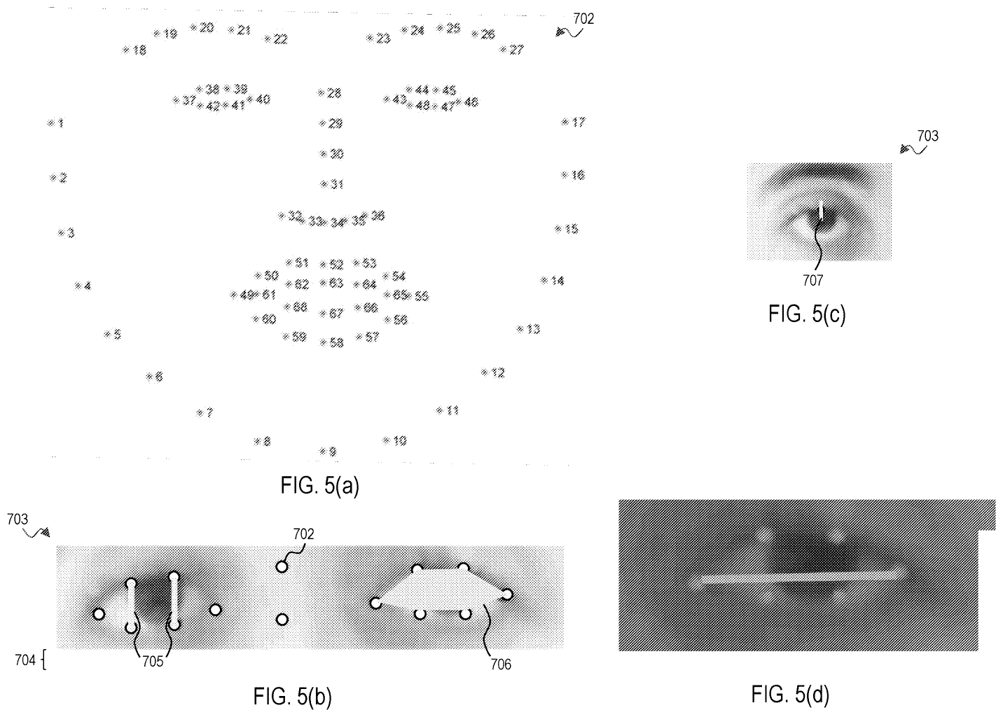

Similarly, the practitioner 102 may ask the patient 101 to perform a first Exercise 1 (look up) and a second Exercise 2, as discussed further below. In those instances, the computer vision module 724 may identify the face and/or eyes of the patient 101 in the patient video data 744 and identify and track face landmarks 702 (e.g., as shown in FIG. 5(a)) to determine if the patient 101 can perform those Exercises. Additionally, the computer vision module 724 may track the movement of those face and/or eye landmarks 702 to determine if the patient 101 experiences ptosis (eyelid droop) or diplopia (double vision) within certain predetermined time periods (e.g., in less than 1 second, within 1 to 10 seconds, or within 11 to 45 seconds). To identify and track face landmarks 702, the computer vision module 724 may use any of a number of commonly used algorithms, such as the OpenCV implementation of the Haar Cascade algorithm, which is based on the detector developed by Rainer Lienhart.

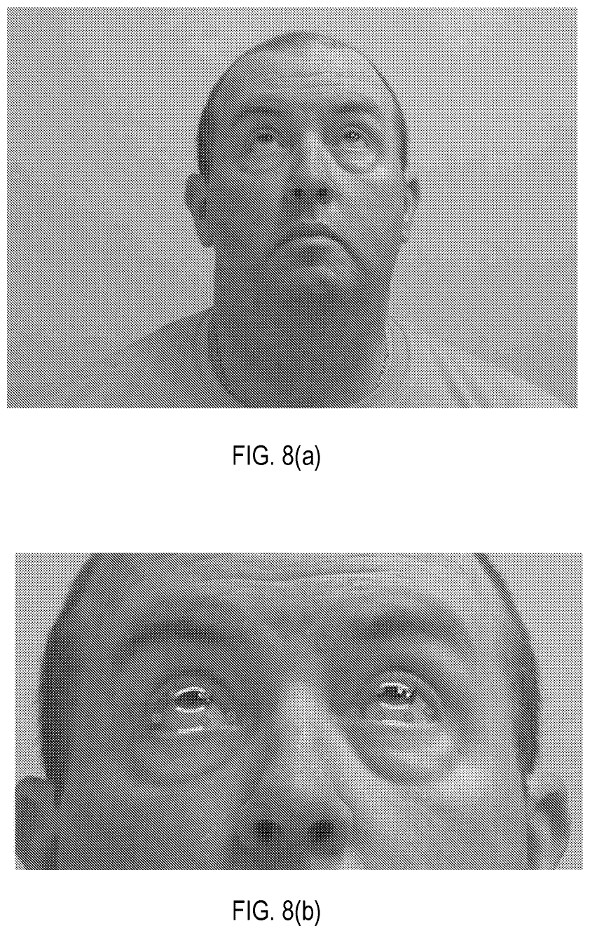

The assessment of diplopia and ptosis will be described in more detail with respect to FIGS. 8-16 below. To assess diplopia, for example, as shown in FIG. 5(b), 5(c), 5(d), the computer vision module 724 may track eye motion to verify the quality of the Exercise, identify the duration of each phase, and register the time stamp of the patient expressing the moment double vision occurs. To assess ptosis, for example, deep learning may be used to identify regions of interest 703 in the patient video data 744, identify face landmarks 702 in those regions of interest 703, and measure eye dimension metrics 704 used in the eye motion assessment, such as the distance 705 between upper and lower eye lid, the area 706 of the eye opening, and the distance 707 from the upper lid to the center of the pupil.

Because the accuracy of the face landmarks 702 may not be adequate to provide accurate enough eye dimension metrics 704 to assess ptosis and ocular motility, however, the cyber-physical system 100 may superimpose the face landmarks 702 and eye dimension metrics 704 identified using deep learning approach over the regions of interest 703 in the patient video data 744 and provide functionality (e.g., via the practitioner user interface 900) to adjust those distances 705 and 707 and area 706 measurements (e.g., after the neurological examination).

As to FIG. 1, the hybrid algorithm for eye tracking that combines deep learning and computer vision can be running at the patient computer to limit the need on the bandwidth or the network and maximize cybersecurity, but the patient computer will need to be powerful enough. This is favored when the telehealth consultation is done at a location where the inteleclinic equipment is provided. In another embodiment, the hybrid algorithm is provided on the doctor computer, but will require that the doctor computer gets the highest possible quality of the video of the patient, to get accurate results, and a good network bandwidth. In another embodiment, the hybrid algorithm is provided in the cloud, such as at a server, in which case a good network bandwidth is needed as in solution two, but cybersecurity is well managed as in solution one.

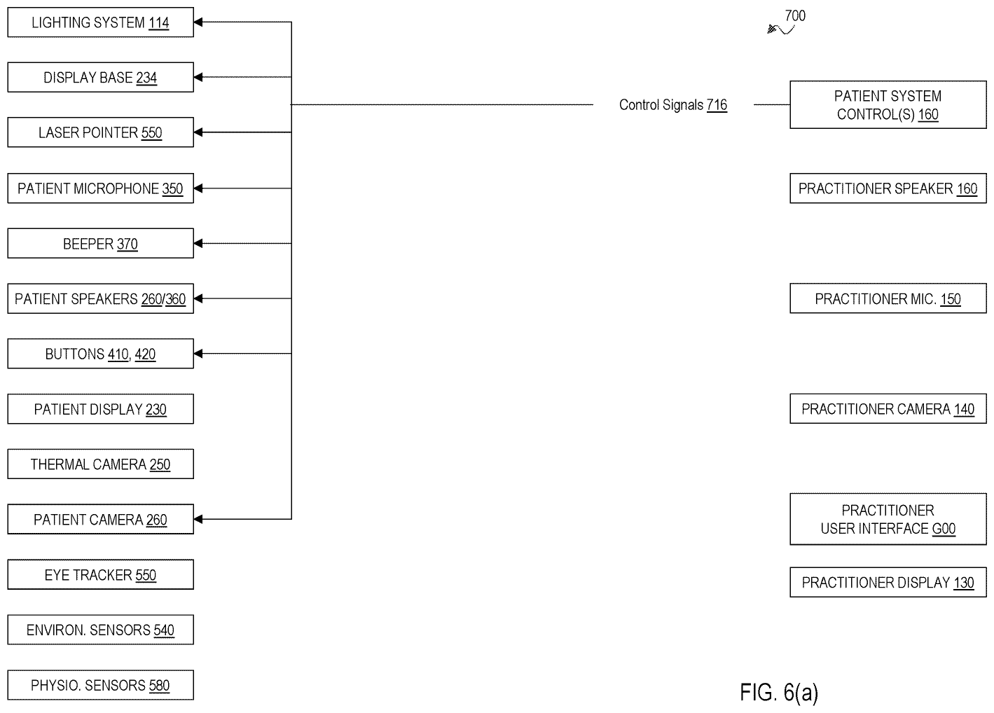

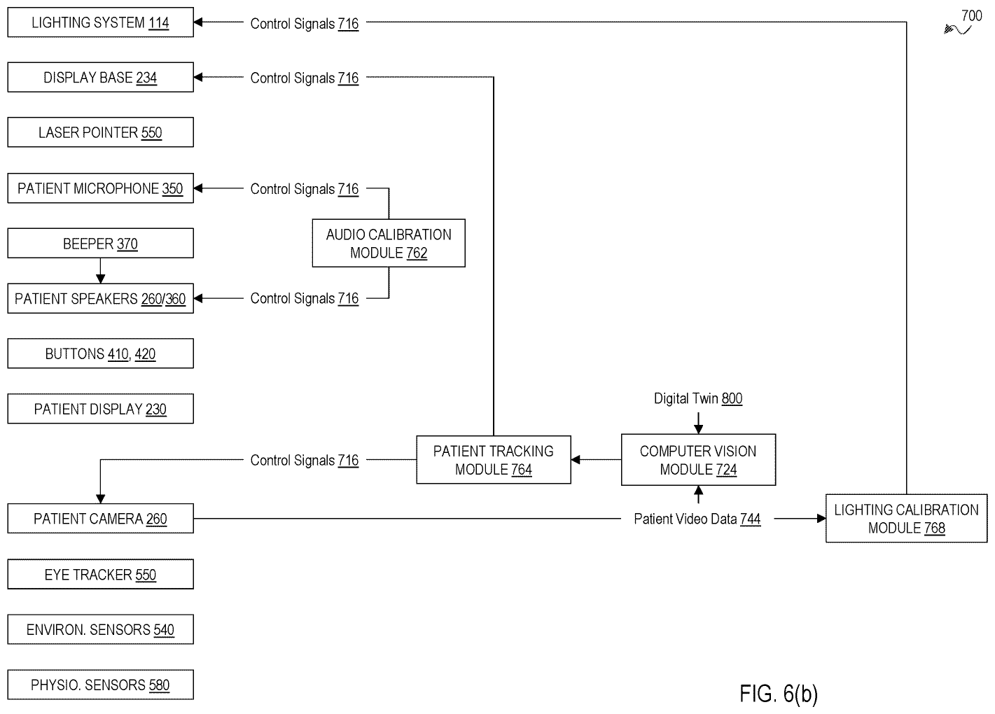

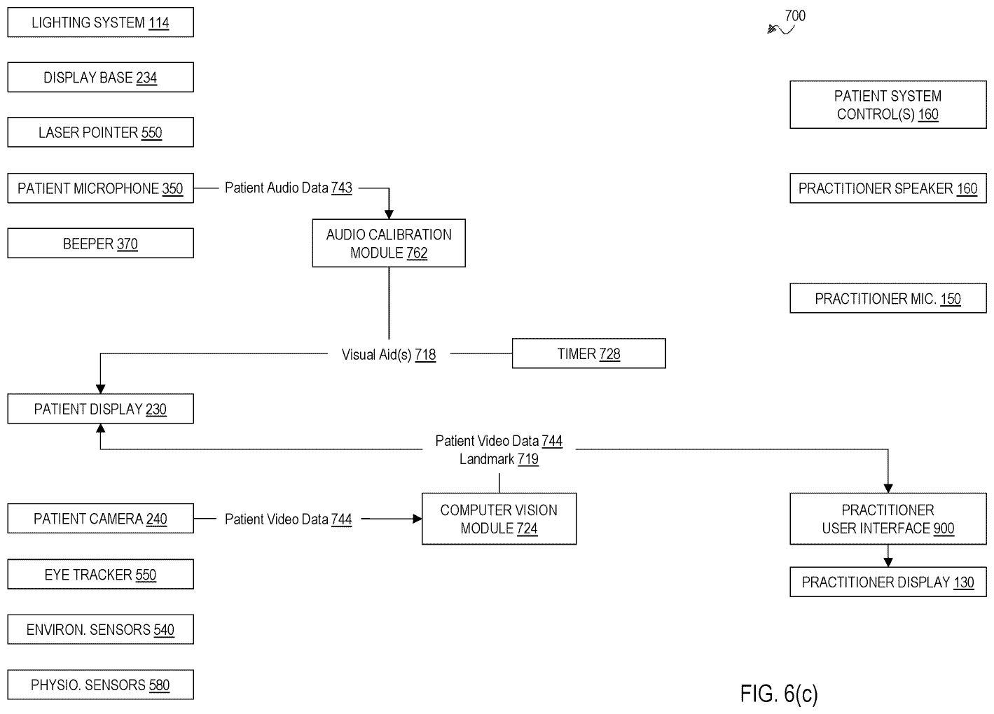

As shown in FIG. 6(a), the cyber-physical system 100 provides patient system controls 160, enabling the practitioner 102 to output control signals 716 to control the pan, tilt, and/or zoom of the patient camera 260, adjust the volume of the patient speakers 260 and/or the sensitivity of the patient microphone 350, activate the beeper 370 and/or illuminate the buttons 410 and 420, activate and control the direction of the laser pointer 550, rotate and/or tilt the display base 234, and/or adjust the brightness of the lighting system 114. The patient system controls 160 may be, for example, a hardware device or a software program provided by the practitioner system 120 and executable using the practitioner user interface 900.

Accordingly, once the telehealth connection is established, the cyber-physical system 100 enables the practitioner 102 to get the best view of the patient 101, zoom in and zoom out in the regions of interest 703 important to the diagnosis, orient the patient display 230 so the patient 101 is well positioned to view the practitioner 102, and control the sound volume of the patient speaker 260 and/or 360, the sensitivity of the patient microphone 350, and the brightness of the lighting in the patient environment 110. Accordingly, the practitioner 102 benefits from a much better view of the region of interest than with an ordinary telehealth system. For example, it would be much more difficult to ask an elderly patient 101 to hold a camera toward the region of interest to get the same quality of view.

As shown in FIG. 6(b), control signals 716 may also be output by an audio calibration module 762, a patient tracking module 764, and/or a lighting calibration module 768. Traditional telemedicine systems can introduce significant variability in the data acquisition process (e.g., patient audio data 743 recorded at an inconsistent volume, patient video data 744 recorded in inconsistent lighting conditions). In order to calculate accurate state variables 810, it is important to reduce that variability, particularly when capturing sensor data 740 from the same patient 101 over multiple telehealth sessions. Accordingly, the cyber-physical system 100 may output control signals 716 to reduce variability in the data acquisition process. For example, the lighting calibration module 768 may determine the brightness of the patient video data 744 and output control signals 716 to the lighting system 114 to adjust the brightness in the patient environment 110.

The patient tracking module 764 may use the patient video data 744 to track the location of the patient 101 and output control signals 716 to the patient camera 260 (to capture images of the patient 101) and/or to the display base 234 to rotate and/or tilt the patient display 230 towards the patient 101. Additionally or alternatively, the patient tracking module 764 may adjust the pan, tilt, and/or zoom of the patient camera 260 to automatically provide a view selected by the practitioner 102 (e.g., centered on the face of the patient 101, capturing the upper body of the patient 101, a view for a dialogue with the patient 101 and a nurse or family member, etc.), or to provide a focused view of interest based on sensor interpretation of vital signs or body language in autopilot mode.

In some embodiments, the patient tracking module 764 automatically adjusts the pan, tilt, and/or zoom of the patient camera 260 to capture each region of interest 703 relevant to each assessment being performed. As shown in FIG. 6(b), for instance, the computer vision module 724 identifies the regions of interest 703 in the patient video data 744 and the patient tracking module 764 outputs control signals 716 to the patient camera 260 to zoom in on the relevant region of interest 703. Generic artificial intelligence and computer vision algorithms may be insufficient identify the specific body parts of patients 101, particularly patients 101 having certain conditions (such as Myasthenia Gravis). However, the cyber-physical system 100 has access to the digital twin 800 of the patient 101, which includes a mathematical representation of biological characteristics of the patient 101 (e.g., eye color, height, weight, distances between body landmarks 701 and face landmarks 702, etc.). Therefore, the digital twin 800 may be provided to the computer vision module 724. Accordingly, the computer vision module 724 is able to use that specific knowledge of the patient 101 (together with general artificial intelligence and computer vision algorithms) to identify the regions of interest 703 in the patient video data 744 so that the patient camera 260 can zoom in on the region of interest 703 that relevant to the particular assessment being performed.

Additionally, to the limit any undesired impact on the emotional and social state of the patient 101 caused by the telehealth session, in some embodiments the cyber-physical system 100 may monitor the emotive state variables 840 and/or social state variables 880 of the patient 101 and, in response to changes in the emotive state variables 840 and/or social state variables 880 of the patient 101, adjust the view output by the patient display 230, the sounds output via the patient speakers 260 and/or 360, and or the lights output by the lighting system 114 and/or the buttons 410 and 420 (e.g., according to preferences specified by the practitioner 102) to minimize those changes in the emotive state variables 840 and/or social state variables 880 of the patient 101.

As shown in FIG. 6(c), the cyber-physical system 100 may also output visual aids 718 to assist the patient 101 and/or the practitioner 102 to capture sensor data 720 using a consistent process. In the Exercises 1, 2 described below, for example, the timer 728 may be used to provide a visual aid 718 (e.g., via the patient display 230) to guide the patient 101 to start and stop an Exercise, or to show the patient the proper technique for conducting the Exercise. Additionally, to ensure that patient audio data 743 is captured at a consistent volume as described above, the audio calibration module 762 may analyze the patient audio data 743 and provide a visual aid 718 to the patient 101 (e.g., in real time) instructing the patient 101 to speak at a higher or lower volume.

Additionally, digitalization of the ptosis, diplopia, and Exercises depends heavily on controlling the framing of the regions of interest 703 (and the distance from the camera patient camera 240 to the region of interest 703). Therefore, the patient video data 744 may be output to the patient 101 (and/or the practitioner 102) with a landmark 719 (e.g., a silhouette showing the desired size of the patient 101) so the practitioner 102 can make sure the patient 101 is properly centered and distanced from the patient camera 240.

FIG. 7 illustrates the practitioner user interface 900 according to an exemplary embodiment. As shown in FIG. 7, the practitioner user interface 900 may include patient video data 644 showing a view of the patient 101, practitioner video data 614 showing a view of the practitioner 102, and patient system controls 160 (e.g., to control the volume of the patient video data 644, control the patient camera 260 to capture a region of interest 603, etc. In the embodiment of FIG. 7, the practitioner user interface 900 also includes a workflow progression 930, which provides a graphic representation of the workflow progress (e.g., a check list, a chronometer, etc.). Additionally, the practitioner user interface 900 provides a flexible and adaptive display of patient metrics 950 (e.g., sensor data 740 and/or state variables 810).

The server 180, the physician system 120, and the compact computer 510 of the patient computing system 500 may be any hardware computing device capable of performing the functions described herein. Accordingly, each of those computing devices includes non-transitory computer readable storage media for storing data and instructions and at least one hardware computer processing device for executing those instructions. The computer processing device can be, for instance, a computer, personal computer (PC), server or mainframe computer, or more generally a computing device, processor, application specific integrated circuits (ASIC), or controller. The processing device can be provided with, or be in communication with, one or more of a wide variety of components or subsystems including, for example, a co-processor, register, data processing devices and subsystems, wired or wireless communication links, user-actuated (e.g., voice or touch actuated) input devices (such as touch screen, keyboard, mouse) for user control or input, monitors for displaying information to the user, and/or storage device(s) such as memory, RAM, ROM, DVD, CD-ROM, analog or digital memory, database, computer-readable media, and/or hard drive/disks. All or parts of the system, processes, and/or data utilized in the system of the disclosure can be stored on or read from the storage device(s). The storage device(s) can have stored thereon machine executable instructions for performing the processes of the disclosure. The processing device can execute software that can be stored on the storage device. Unless indicated otherwise, the process is preferably implemented automatically by the processor substantially in real time without delay.

The processing device can also be connected to or in communication with the Internet, such as by a wireless card or Ethernet card. The processing device can interact with a website to execute the operation of the disclosure, such as to present output, reports and other information to a user via a user display, solicit user feedback via a user input device, and/or receive input from a user via the user input device. For instance, the patient system 200 can be part of a mobile smartphone running an application (such as a browser or customized application) that is executed by the processing device and communicates with the user and/or third parties via the Internet via a wired or wireless communication path.

The system and method of the disclosure can also be implemented by or on a non-transitory computer readable medium, such as any tangible medium that can store, encode or carry non-transitory instructions for execution by the computer and cause the computer to perform any one or more of the operations of the disclosure described herein, or that is capable of storing, encoding, or carrying data structures utilized by or associated with instructions. For example, the database 182 is stored is non-transitory computer readable storage media that is internal to the server 180 or accessible by the server 180 via a wired connection, a wireless connection, a local area network, etc.

The heuristic computer reasoning engine 890 may be realized as software instructions stored and executed by the server 180. In some embodiments, the sensor data classification module 720 may be realized as software instructions stored and executed by the server 180, which receives the sensor data 740 captured by the patient computing system 500 and data (e.g., input by the physician 102 via the physician user interface 900) from the physician computing system 102. In preferred embodiments, however, the sensor data classification module 720 may be realized as software instructions stored and executed by the patient system 200 (e.g., by the compact computer 510 of the patient computing system 500). In those embodiments the patient system 200 may classify the sensor data 740 (e.g., as belonging to one of a number of predetermined ranges and/or including any of a number of predetermined patterns) using algorithms (e.g., lower-level artificial intelligence algorithms) specified by and received from the server 180.

Analyzing the sensor data 740 at the patient computing system 500 provides a number of benefits. For instance, the sensor data classification module 720 can accurately time stamp the sensor data 740 without being affected by any time lags caused by network connectivity issues. Additionally, analyzing the sensor data 740 at the patient computing system 500 enables the sensor data classification module 720 to analyze the sensor data 740 at its highest available resolution (e.g., without compression) and eliminates the need to transmit that high resolution sensor data 740 via the communications networks 170. Meanwhile, by analyzing the sensor data 740 at the patient computing system 500 and transmitting state variables 810 to the server 180 (e.g., in encrypted form), the cyber-physical system 100 may address patient privacy concerns and ensure compliance with regulations regarding the protection of sensitive patient health information, such as the Health Insurance Portability and Accountability Act of 1996 (HIPAA).

### Deep Learning and Computer Vision Overview

The present disclosure assesses quantitatively anatomic metrics during a telehealth session such as, for example, ptosis, eyes misalignment, arms angle, speed to stand up, lip motion. This anatomic metric can be from a single image at some specific time, or a video. For video, the system also looks for a time variation of the anatomic metric. The system uses a deep learning library to compute these anatomic metrics. Off-the-shelf libraries are available, such as for example from Google or Amazon. However, these AI algorithms (deep learning algorithms) have not been trained for specific anatomic metrics, such as for example, ptosis where the patient eye is looking up, and diplopia where the patient is looking sideways or undergoing MG examination, such as for example described in A. Guidon, S. Muppidi, R. J. Nowak, J. T. Guptill, M. K. Hehir, K. Ruzhansky L. B., Burton, D. Post, G. Cutter, R. Conwit, N. I. Mejia, H. J. Kaminski, J. F. Jr. Howard, Telemedicine visits in myasthenia gravis: Expert guidance and the myasthenia gravis core exam (MG-CE) Muscle Nerve 2021; 64:270-76.

Consequently, though those deep learning algorithms are robust, they are not precise enough for medicine nor come with error estimates that would make them secure to use. Accordingly, the present system starts with the markers provided by the AI algorithm (i.e., the deep learning algorithms), which are shown for example by the dots in FIGS. 8(b), 9(b), 11(a), 11(b), 13(a), 13(b). The system then uses computer vision to localize precisely each anatomic marker, which are shown for example by the lines in FIGS. 8(b), 9(b), 11(a), 11(b), 13(a), 13(b).

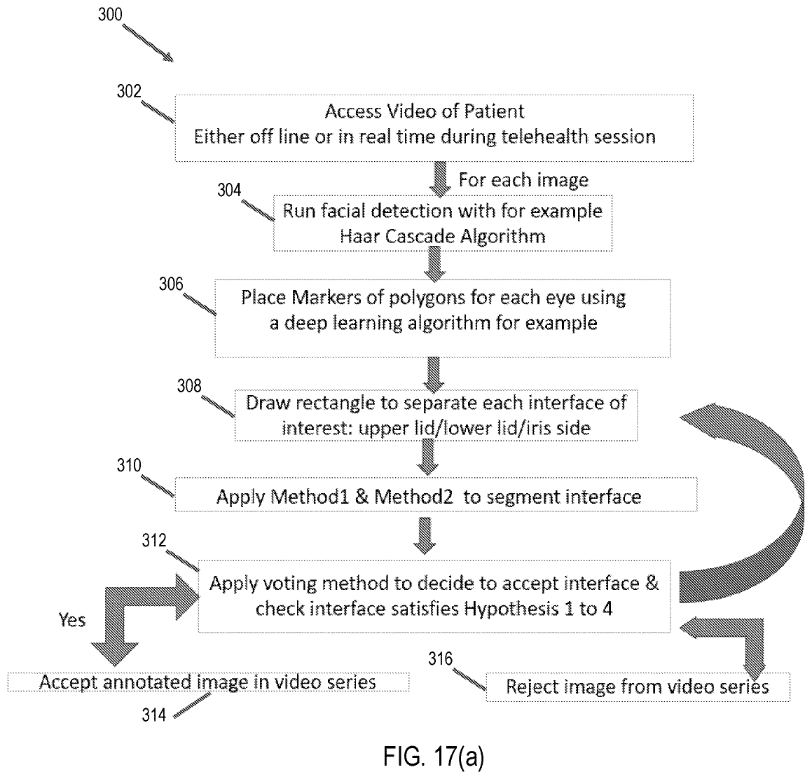

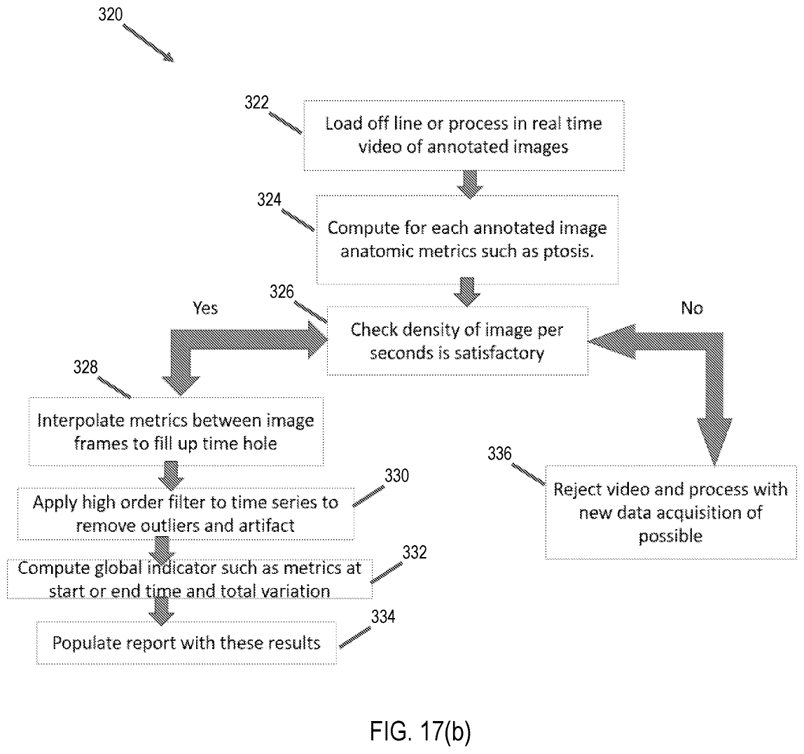

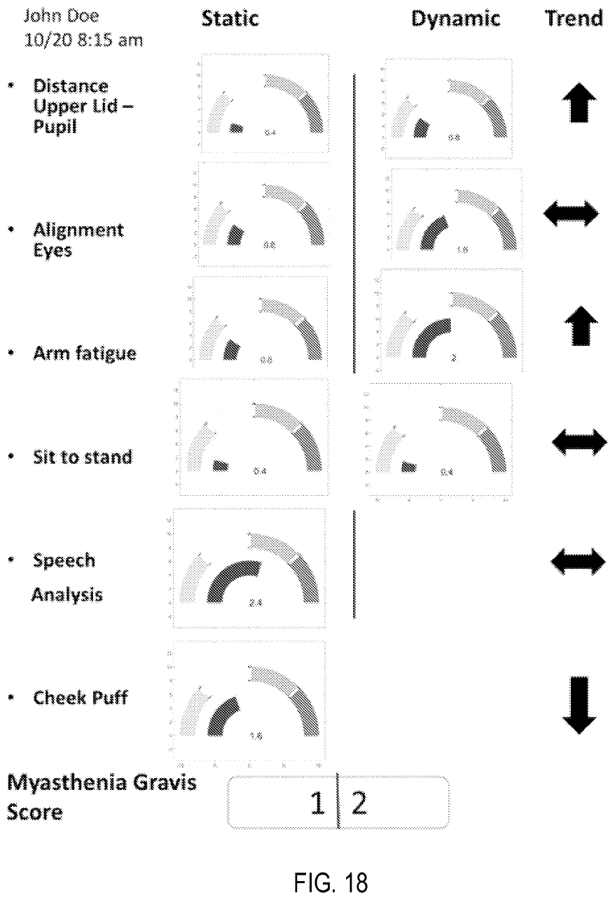

The overall operation 300, 320 of the system is shown in a non-limiting illustrative example embodiment, in FIGS. 17(a), 17(b), which will be described more fully below. As illustrated, deep learning is performed at steps 304, 306, and computer vision is performed at step 310. Step 308 transitions from deep learning to local computer vision precision edit of interfaces of interest as requested. FIG. 17(b) is only the postprocessing piece that provide the metrics and populates the report once the hybrid algorithm (i.e., deep learning followed by computer vision) has done the job. Once the annotated images are accepted at step 314, those annotated images are used at step 322. One result of postprocessing is to generate a report, step 334, such as shown in FIG. 18.

The deep learning algorithms can be implemented by transmitting data from either a processing device 510 at the patient system 200 and/or the practitioner system 120, to a remote processing device, such as at the server 180, and the library stored at the database 182. In other embodiments, the deep learning can be implemented at the patient's processing device 510 or the practitioner's system 120, such as by a processing device at the practitioner's system 120.

The computer vision can be implemented at the practitioner's system 120, such as by a processing device at the practitioner's system 120. In other embodiments, the computer vision can be implemented patient's processing device 510, or by transmitting data from either a processing device 510 at the patient system 200 and/or the practitioner system 120, to a remote processing device, such as at the server 180.

### Ptosis and Diplopia

As noted below, the system 100 is utilized to detect eye position to determine ptosis and diplopia, which in turn can signify MG. The NIH Rare Disease Clinical Research Network dedicated to myasthenia gravis (MGNet) initiated an evaluation of examinations performed by telemedicine. The study recorded the TM evaluations including the MG Core Exam (MG-CE) to assess reproducibility and exam performance by independent evaluators. These Zoom recordings performed at George Washington University, were utilized to evaluate the technology. Two videos of each subject were used for quantitative assessment of the severity of ptosis and diplopia for patients with a confirmed diagnosis of myasthenia gravis. The patients were provided instructions regarding their position in relationship to their cameras and levels of illumination as well as to follow the examining neurologist's instructions on performance of the examinations.

In Exercise 1 of the MG-CE, the patient must hold his gaze up for 61 seconds, see FIGS. 8(a), 8(b). The goal is to assess the severity of ptosis (uncontrolled closing of eyelid), if any, before and after the Exercise [14] ratings: (0) for no visible ptosis within 45s; (1) for visible ptosis within 11-45 s; (2) for visible ptosis within 10 s; and (3) for immediate ptosis. Another grading system was for the MG-CE using the following ratings: (0) for no ptosis; (1) for mild, eyelid above pupil; (2) for moderate, eyelid at pupil; and (3) for severe, eyelid below pupil.

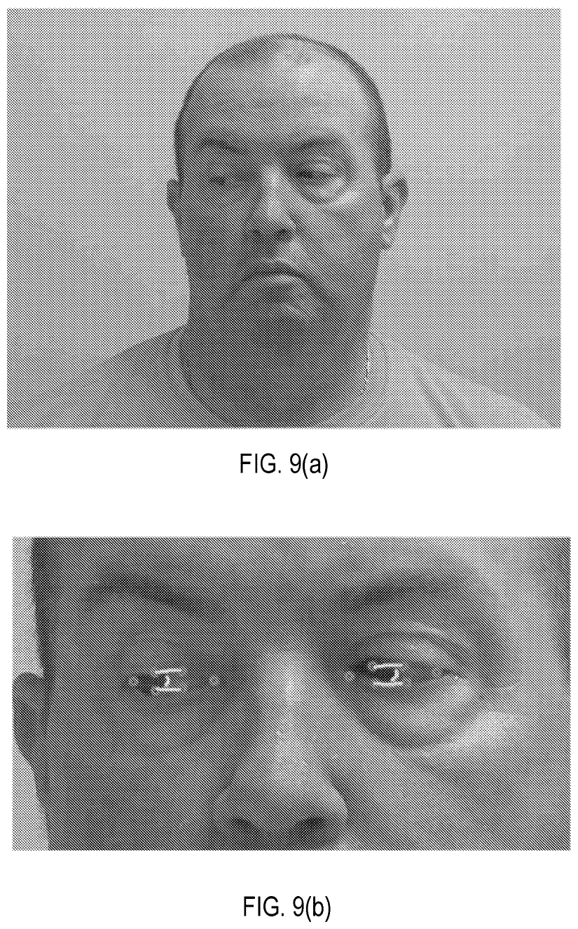

In Exercise 2 of the MG-CE, the patient must hold his gaze right and left respectively for 61 seconds, see FIG. 9. The goal is to check for diplopia (double vision), and when it appears. Ratings range from 0 to 3: (0) for no diplopia with 61 s sustained gaze; (1) for diplopia with 11-60 s sustained gaze; (2) for diplopia within 1-10 s but not immediately; and (3) for immediate diplopia with primary or lateral gaze.

As noted above, the system 100 can be utilized to automatically administer one or more Exercises to the patient 101, who performs the Exercises at the patient system 200. For example, the system 100 can display the appropriate technique in a video or written instructions to the patient, and can indicate if the patient isn't performing the Exercise correctly. For example, if the user is performing Exercise 1, the system 100 can indicate the start and stop time for the Exercise, and if the system 100 detects that the patient isn't looking up, the system 100 can indicate that to the patient.

One goal is to take accurate and robust measurements of the eye anatomy in real-time, during the Exercises, and automatically grade possible ptosis and ocular misalignment. The algorithm should reconstruct the eye geometry of the patient from the video and the position of the pupil inside that geometric domain. The difficulty is to precisely recover those geometric elements from a video of the patient where the eye dimension in pixel is about 1/10 of the overall image dimension, at best. Most of the studies of oculometry assume that the image is centered on the eye that occupied most of the image. Alternatively, eye trackers do not rely on standard camera using the visual spectrum but rather use infrared in order to isolate clearly the pupil as a feature in the corneal reflection image [15,16,17].

Presently, localization of eye position can take advantage of deep learning methods but requires large, annotated data sets for training [18,19]. From a model of eye detection, the system can focus the search for pupil and iris location in the region of interest [20]. Among the popular techniques to detect the iris location [21] are the circular Hough transform [22,23] and the Daughman's algorithm method [24].

Systems having a standard camera that operates in the visual spectrum, have a robustness issue due to their sensitivity to low resolution of the eyes' Region Of Interest (ROI), poor control on illumination of the subject, and specific eye geometry consequent to ptosis. The present system and method is a hybrid that combines existing deep learning library for face tracking and a local computer vision system to build ptosis and diplopia metrics. The deep learning (steps 302-306, FIG. 17(a)) provides a coarse identification of the ROI for the eyes, and the computer vision system (steps 308-310) fine tunes that coarse identification and corrects for any errors in the coarse identification, and provides a final ROI identification for the eyes.

One goal of the present system is to take accurate and robust measurements of the eye anatomy in real-time, during the Exercises, and automatically grade possible ptosis and ocular misalignment. The algorithm reconstructs the eye geometry of the patient from the video and the position of the pupil inside that geometric domain. The difficulty is to precisely recover those geometric elements from a video of the patient where the eye dimension in pixel is about 1/10 of the overall image dimension, at best. Most of the studies of oculometry assume that the image is centered on the eye that occupied most of the image. Alternatively, eye trackers do not rely on standard camera using the visual spectrum but rather use infrared in order to isolate clearly the pupil as a feature in the corneal reflection image [15,16,17].

Presently, localization of eye position can take advantage of deep learning methods but requires large, annotated data sets for training [18,19]. From a model of eye detection, the present system 100 can focus the search for pupil and iris location in the region of interest [20]. Among the popular techniques to detect the iris location [21] are the circular Hough transform [22,23] and the Daughman's algorithm method [24].

The system 100 was tested with 12 videos acquired by Zoom during the ADAPT study telehealth sessions of 6 patients with MG. Each subject had TM evaluations within 48 hours of each other and participated in a set of standardized outcome measures including the MGNet Core Exam [5]. Telehealth session were organized as Zoom meetings by a board-certified neurologist with subspecialty training in neuromuscular disease in the clinic providing the assessments of all patients at their homes. In practice, these Zoom sessions were limited in video quality to a relatively low resolution in order to accommodate the available internet bandwidth and because they were recorded on the doctor side during streaming. We extracted fixed images at various steps of the Exercise to test the system 100 and algorithm, as well as on video clips of about 60 seconds each for each Exercise 1 and 2 described above. The number of pixels per frame was as low as 450*800 at a rate of 30 Frames Per Second (FPS).

The distance from the patient to the camera and illumination of the subject led to variability of the evaluations. Those conditions are inherent limitations of the telehealth standard to accommodate patients' equipment and home environment. We also included half a dozen video of healthy subjects acquired in the same conditions than the ADAPT patients.

The system 100 includes a high resolution camera, here a Lumens B30U PTZ camera 240 (Lumens Digital Optics Inc., Hsinchu, Taiwan) with a resolution of 1080*1920 at 30 FPS, which is plugged into a Dell Optiplex 3080 small form factor computer (Intel processor i5-10500t, 2.3 GHz, 8 Gb Ram) where the processing is done. This system, tested initially on healthy subjects, was used eventually on one patient following the ADAPT protocol. We have acquired through this process a data set that is large enough to test the robustness and quality of the algorithms. Error rates depending on resolution and other human factors were compared.

### Face and Eyes Detection

Before the system can detect eye conditions, the system must first detect the patient's eyes in the image. Accordingly, with reference to FIGS. 4, 17(a), the system 100 detects the face in the image. As discussed above, in one embodiment, the patient camera 240 captures patient video data, either offline or in real time during a telehealth session, step 302, and sends that to the sensor data classification module 720, which can either be located either at the videoconferencing module 710, the patient system 200 or the practitioner system 120. At step 304, the classification module 720 can use deep learning to identify the landmark points 702 for the face and/or eyes (FIG. 5(a)). This can be accomplished in any suitable manner, such as for instance any of the multiple face tracking algorithms and compared methods for face detection [25,26]. Among the most widely used algorithms, the system uses OpenCV's implementation of the Haar Cascade algorithm [27], based on the detector from R. Lienhart [28] that is a fast method and overall most reliable for real-time detection.

Once face and eye detection have been confirmed through deep learning, the system can then be utilized to compute ptosis utilizing computer vision. Thus, once a bounding box of the face is detected, key facial landmarks are required to monitor the patient's facial features. Thus, at step 306, markers of polygons are placed for each eye using the deep learning algorithm. Those markers are used for the segmentation and analysis portion of computer vision to evaluate weakness of MG. In principle, these interface boundaries should cross horizontally the rectangle for lid position, respectively and vertically for ocular misalignment. Thus, at step 308, a rectangle is determined (and can be drawn on the display), to separate each interface of interest, such as for example, the upper lid and lower lid, and the iris side.

The system checks with an algorithm that the interface partitions the rectangle into two connex sub domains. At step 310, the segmentation algorithm may shrink the rectangle to a smaller dimension as much as necessary to separate each anatomic feature. For example, to position the lower lid and the lower boundary of the iris during the ptosis exercise 1. To improve the lower lid positioning, the system draws a small rectangle (step 308) including the landmark points (42) (41) and looks for the interface (steps 310, 312) between the sclera and the skin of the lower lid. Similarly, the system draws a rectangle that contains (38) (39) (40) (41) and identify the interface of the iris and sclera.

For face alignment, many methods exist. Some of these image-based techniques were reviewed by Johnston and Chazal [29]. One of the most time-efficient for real-time application is based on the shape regression approach [30]. The system uses DLib's implementation of the regression tree technique from V. Kazemi and J. Sullivan [31] which was trained on the 300 W dataset [32] fitting a 68 points landmark to the face (FIGS. 5(a), 5(b)). The ROI for each eye is the polygon formed by the points 37 to 42 with the right eyes, respectively 43 to 48 with the left eyes in reference to the model in FIG. 5(a). FIG. 5(a) is the model that is used in the off the shelf deep learning library. The face is given by the polygon joining points 1 to 27. The left eye is the polygon joining points 43 to 48, and the right eye is the polygon joining the points 37 to 42.

### Computing the Ptosis Metrics:

First, the system processes the time window of the video clip when the patient is executing the first Exercise (Exercise 1) maneuver, i.e., focusing eye gaze up.

The ROI for each eye enables the system to determine a first approximation of ptosis, such as based on Exercise 1 of the MG-CE. FIG. 5(b) shows the eyelid opening distance for the patient's right eye (on the left in the embodiment of FIG. 5(b)). The system 100 determines an eyelid opening distance (ED) approximation as the average distance between respective points of the upper eyelid (see FIG. 5(a), segments 38-39 for the right eye, and respectively segments 44-45 for the left eye) and respective points on the lower eyelids (segment 42-41 for the right eye, respectively segments 48-47 for the left eye).

The deep learning algorithm using the model of FIG. 5(a) corresponds to step 306 (FIG. 17(a)). But to run the deep learning model, an initial other AI algorithm is needed to localize the face in the video. This is a very rough localization that simply draws a box around the face and does not have all the details of FIG. 5(a), step 304. FIG. 17(b) uses the output of step 314 to construct a report, which has many algorithmic steps to provide an accurate result and interpret that result.

That is, the average distance is taken between respective points on the upper and lower eyelids, for each the right eye and the left eye. Thus, for the right eye, a first right eye distance is taken from segment 38 (right center of the upper eyelid for the right eye) and segment 42 (right center of the lower eyelid for the right eye); and a second right eye distance is taken from segment 39 (left center of the upper eyelid for the right eye) and segment 41 (left center of the lower eyelid for the right eye). For the left eye, a first left eye distance is taken from segment 44 (right center of the upper eyelid for the left eye) and segment 48 (right center of the lower eyelid for the left eye); and a second left eye distance is taken from segment 45 (left center of the upper eyelid for the left eye) and segment 47 (left center of the lower eyelid for the left eye). An average eye open distance is then determined based on the first and second right eye distances and first and second left eye distances.

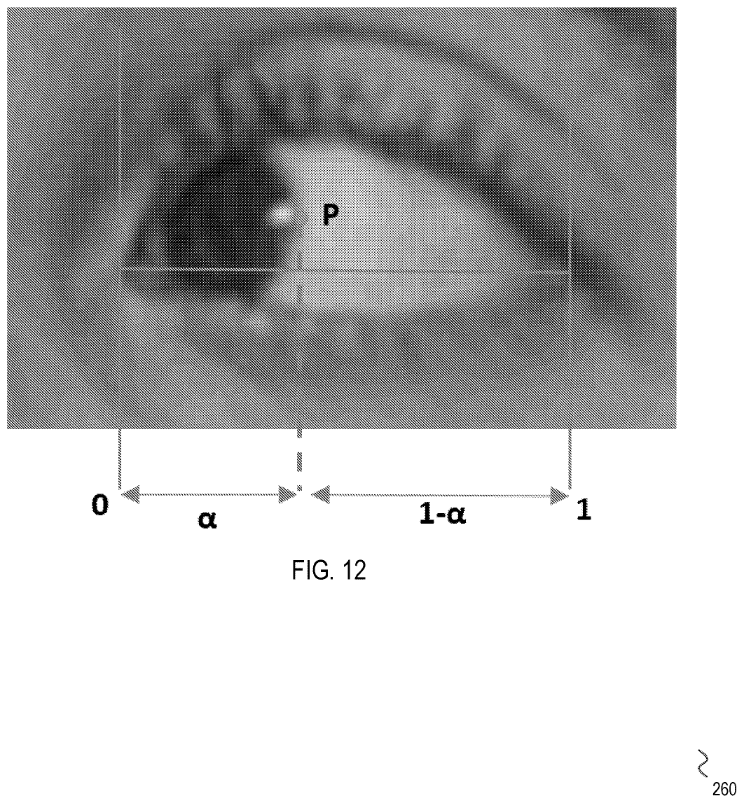

The system computes eye misalignment and ptosis as distance between interfaces, i.e., curves. For ptosis, it is defined as the maximum distance between the upper lid and lower lid along a vertical direction. For diplopia, the system uses a comparison between the barycentric coordinates of the iris side in each eye, FIG. 12.

FIG. 5(b) also shows eye area for the patient's left eye. The system determines the eye area, which is the area contained in the outline of the eye determined by the landmark points 37-42 (right eye) and 43-48 (left eye) (FIG. 5(a)). The system normalizes these measurements by the eye length (EL), as the horizontal distance between the two eye corners' landmark points 37, 40 (right eye) and 43, 46 (right eye), as illustrated in FIG. 5(d). Any distance metric on ptosis used in the report is divided by a characteristic dimension of the eye (distance between left and right corner) in that way the metric is independent of the distance between the subject and the camera.

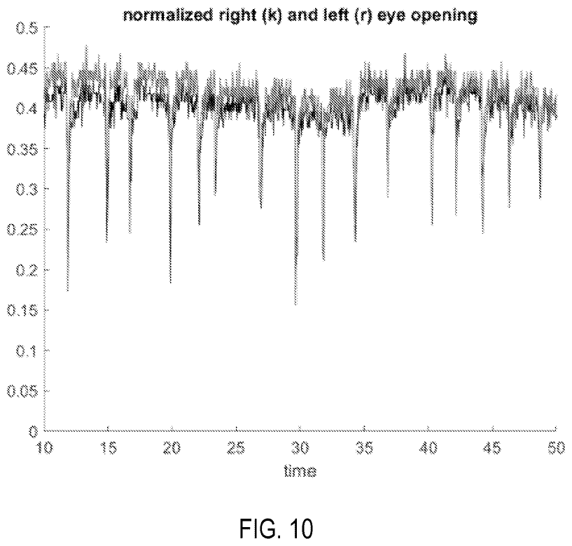

In addition, the system determines the blink rate, if any, FIG. 10. The system detects eye blinking when each lower pick for the right and left eye openings are perfectly synchronized. The system can then determine the blink rate of eye blinking, and if there is a neurological disease, since a neurological disease can give abnormal blink rates.

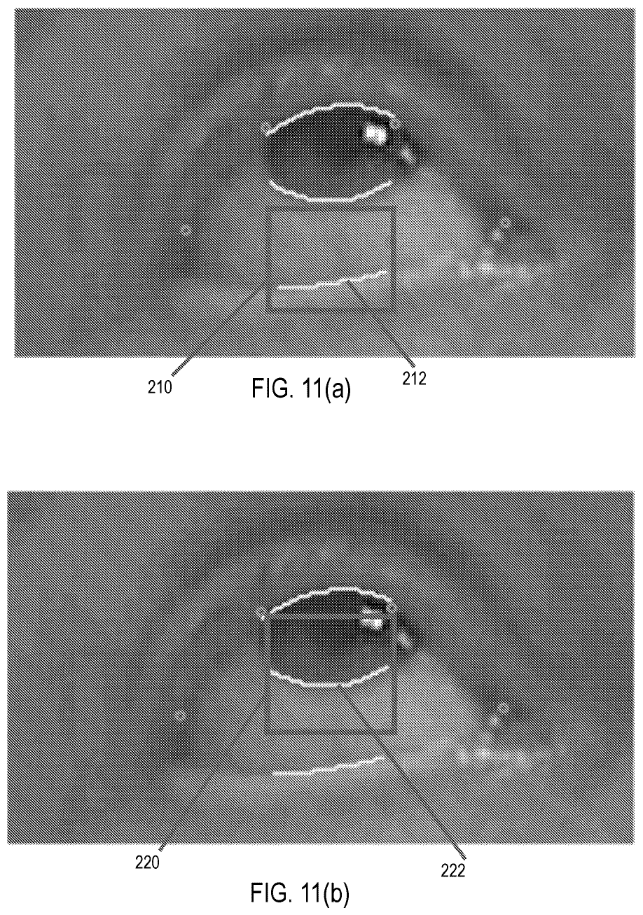

As shown in FIGS. 11(a), (b), the eye lid location provided by the deep learning algorithm may not be accurate. For example, in FIGS. 11(a), (b), the lower landmarks (41) and (42) are quite off the contour of the eye, and the landmarks (37) and (40) are not quite located at the corner of the eye. The accuracy of the deep learning library varies depending on the characteristic of the patient, such as iris color, contrast with sclera, skin color, etc. The accuracy also depends on the frame of the video clip and potential effect of lightning or small variation of head position.

Under optimal conditions, the landmark points 37-42 and 43-48 form a hexagon shape; for example, the right eye hexagon has a first side 37-38, second side 38-39, third side 39-40, fourth side 40-41, fifth side 41-42, and sixth side 42-37. However, the hexagon of the model found by the deep learning algorithm may degenerate, such as to a pentagon, when a corner point overlaps another edge of the hexagon (which has 6 edges). In extreme cases, the ROI can be at the wrong location altogether, e.g., the algorithm confuses the nares with the eye location. Such error is relatively easy to detect but improving the accuracy of the deep learning library for a patient exercising an eccentric gaze position, e.g., as Exercises 1 and 2, would require re-training the algorithm with a model having a larger number of landmarks concentrating on the ROI.

Many eye detections methods have been developed in the field of ocular motility research, but they rely on images taken in a controlled environment with specific infrared lights allowing for a better contrast of the eye and focused on the eye directly.

The system 100 and method of the present disclosure is able to compensate for an inaccurate eye ROI. The system 100 starts from the inaccurate ROI, i.e., the polygons provided by deep learning that is relatively robust with standard video. The system 100 then uses local computer vision algorithms that target special features such as upper lid/lower lid curves, iris boundary of interest for ptosis and diplopia metrics, and pupil location to improve the eye ROI identification. Thus, the deep learning is robust in the region of interest but may lack accuracy; whereas computer vision is best at local analysis in the region of interest but lacks robustness.

The local search positions the lower lid and the lower boundary of the iris during the ptosis Exercise 1, i.e., as the user is looking up, as shown in FIGS. 11(a), (b). Though the description here is with respect to the right eye, the processing of left eye being entirely similar. As shown in FIG. 11(a), to improve the lower lid positioning of the ROI bounding box, the system draws a first rectangle or lower lid rectangle 210 that includes the landmark points (42) (41), step 308 (FIG. 17(a)). In the embodiment shown, points 41, 42 are included in the rectangle 210, whereas points 37, 40 were not; though in other embodiments, points 37, 40 could also be included in the rectangle 210. The system then identifies the lower lid by detecting the lower lid interface 212 between the sclera (i.e., the white of the eye) and the skin that corresponds to the location of the lower lid, step 310. In one embodiment, the interface 212 can be used to determine the bottom of the rectangle 210; though in other embodiments the interfaces 210, 222 can be used to draw the rectangles 210, 220, or the rectangles 210, 220 can be used to identify the interfaces 212, 222.

Referring to FIG. 11(b), the system 100 also draws a second rectangle or iris rectangle 220 that contains landmark points (38) (39) (40) (41) and determines the lower iris interface 222 between the iris (i.e., the colored part of the eye) and the sclera. At step 312, each of the interfaces 212, 222 found by the computer vision algorithm are only acceptable if it is a smooth curve (first condition or hypothesis, H1) that crosses the respective rectangle 210, 220 horizontally (second condition or hypothesis, H2). For the iris bottom interface 222, the curve should also be convex (third condition or hypothesis, H3). The iris is a disc, so it's bottom part, (i.e., curves below the horizontal level of the pupil) is convex, it cannot be straight.

At step 312, a voting method is applied to decide whether or not to accept the interface, and check if the interface satisfies H1-H4. Here, voting uses two different methods from step 310 to compute an interface, or more precisely a specific point that is used to compute the metrics. If both methods agree on the same point, the result of the vote is yes and the choice of that point is considered to be true and the annotated image is accepted and retained in the video series, step 314. If both methods give two points far away, the system cannot decide, so that the vote for any of these two points is no and the image is rejected and removed from the video series, step 316. It is noted that more than two methods can be utilized, and the vote can depend, for example, on whether two (or all three) methods agree on the same point.

At this point, the computer vision is concentrated in a rectangle of interest 210, 220 that contains essentially the interface 212, 222 the system is looking for. So, the problem is simpler to solve and the solution is more accurate. By enhancing the contrast of the image in that rectangle 210, 220, further processing is simpler and very efficient. The system utilizes several simple techniques, such as kmeans, restricting to two clusters, or open snake that maximize the gradient of the image along a curve. Those numerical techniques come with numerical indicators to show how well two regions are clearly separated in a rectangular box. The image segmentation automatically finds and draws the line 212.

For example, with the kmean algorithm, the system likes to have the center of the two clusters clearly separated, and each cluster should be a convex set (fourth hypothesis, H4). For the open snake method, the system can check on the smoothness of the curves and the gradient value across that curve.

If the computer vision algorithm (applied at the computer vision module 724) fails to find an interface that satisfies all hypotheses (H1) to (H4), step 312, the system 100 either reruns the k-means algorithm changing the seed, or eventually shrinks the size of the rectangle until convergence to an acceptable solution, step 308. If the computer vision algorithm fails, the system cannot conclude on the lower lid and upper lid position and must skip that image frame in its analysis, step 316.

In the example of FIG. 11, the model provides the correct location of the upper lid, also the contrast between the iris and skin right above is clear. The system uses the local computer vision algorithm only to check the landmark positions.

Overall, the hybrid algorithm combines deep learning with local computer vision technic output metrics such as the distance between the lower lid and the bottom of the iris, the lower lid and the upper lid. The first distance is useful to check that the patient does the Exercise correctly, the second distance provides an assessment of ptosis. It is straightforward to get the diameter of the iris as the patient is looking straight and the pupil should be at the center of the iris circle.

### Computing the Diplopia Metric

As illustrated in FIG. 12, the system uses a similar approach as with respect to FIG. 11, to identify the upper lid and lower lid positions. The only difference here is to identify the correct side boundary of the iris as the patient is looking left or right, using a computer vision algorithm in a small horizontal box that start from the corner of the eye landmark (37) or (40) and goes all the way to the landmarks of the upper lid and lower lid on the opposite side, i.e., (39) and (41) or (38) and (42). The same algorithm is applied to the right eye as described above and left eye.

The system then can compute the barycentric coordinate denoted a of the point P that is most inside point of the iris boundary as shown in FIGS. 9(a), 9(b). The distance from the face of the patient to the camera is much larger than the dimension of the eye and makes the barycentric coordinate quasi-invariant to the small motion of the patient head during the Exercise.

In principle, P_{left} and P_{right} should be of the same order as the subject is looking straight at the camera. α_{left} and α_{right} should also be strongly correlated as the subjects direct their gaze to the side. P_{left} is the left end of the segment in FIG. 12, P_{right} is the right end of the segment in FIG. 12, α_{leg} is alpha, and α_{right} is 1-alpha.

As fatigue occurs, the difference between α_{left} and α_{right} may change with time and corresponds to the misalignment of both eyes. The system determines that diplopia occurs when the difference between α_{left}−α_{right} deviates significantly from its initial value at the beginning of the Exercise. A significant deviation for an interface location can be, for example, a difference of 1-2 pixels would indicate no diplopia, whereas a difference of five or more pixels would be considered a significant difference and that there is diplopia. An iris is typically from 10-40 pixels depending on resolution, so a deviation of over approximately 10% of alpha is considered significant, and especially a deviation of over approximately 20% of alpha is considered significant.

### Eye Gaze and Reconstruction of Ptosis and Diplopia Metrics in Time

We have described so far, the hybrid algorithm (i.e., deep learning to establish the initial landmark points, and computer vision to fine tune those landmark points) that the system runs for each frame of the video clip during Exercise 1 and 2. Now referring to the reporting operation 320 of FIG. 17(b), the system 100 generates a report. As in step 302, the system 100 loads offline or in real time, video of annotated images (i.e., with all the deep learning dots and computer vision lines of FIGS. 8(b), 9(b)), step 322.

At step 324, the computes for each annotated image, anatomic metrics, such as for example ptosis. The system 100 uses a clustering algorithm in the ROI for each eye to reconstruct the sclera area and detect the time window for each Exercise: the sclera should be one side left or right of the iris in Exercise 2 and one side below the iris in Exercise 1 (i.e., the patient is asked to look first on his right side for one minute without moving his head and then on his left side for one minute without moving his head). For each side corresponds a specific side of the iris that the system uses to compute the barucenter coordinates. All the output is displayed in a report (FIG. 18).

Since the system knows a priori that each Exercise lasts one minute, it does not need an extremely accurate method to reconstruct when the Exercise starts or ends. Besides, and for verification purpose, the result on left eye gaze and right eye gaze should be consistent.

Further the computer vision algorithm does not always converge for each frame. So the system 100 can use one or more sensors (e.g., sensors 540, 550, 580) to check for Stability (the patient should keep his/her head in about the same position), Lightning defect (the k-means algorithm shows non-convex clusters in the rectangle of interest when reflecting light affect the iris for example), Instability of the deep learning algorithm output (when the landmarks of the ROI change in time independently of the head position), and Exception with quick motion of eyes due to blinking or reflex that should not enter the ptosis or diplopia assessment. The sensor data classification module 720 (FIG. 4(b)) can receive the sensor data and determine stability, lighting, etc.

At step 326, the density of an image per second is analyzed. Let's say there are 32 image per seconds in the video of one minute for the diplopia exercise. This is about 1800 images. If 30% of the images have been rejected by the algorithm of FIG. 17(a), then there are about 540 images that are missing. If 540 consecutive images are missed, there is a hole in the time series of 20 seconds, which is a big hole that cannot be fixed, so the video is rejected, step 336. However, if there are 10 images per seconds missing out of 32 images per second in the same window of one second, there is no impact at all, since there are many holes of one-third of a second. In one embodiment, if the system does not miss more than 60 images in a row, i.e., 2 seconds, it has enough data to compute the report metrics that has to do with time, which is shown in right column of the report in FIG. 18. The system can then interpolate metrics between the image frames to fill up time holes, step 328.

The system 100 can automatically eliminate all the frames that do not pass these tests, and generate a time series of measures for ptosis and diplopia during each one-minute Exercise that is not continuous in time, for example, using linear interpolation in time to fill the holes provide that the time gap are small enough i.e., a fraction of a second, step 328. All time gaps that are larger than a second are identified in the time series and may correspond actually to a marker of subject of fatigue.

To get the dynamic of the ptosis and diplopia measure that is not part of the standard core exam and present some interest for neuromuscular fatigue, the system 100 post-processes further the signal with a special high order filter as in [35] that can take advantage of Fourier technique for nonperiodic time series, step 330 (FIG. 17(b)).

### Results

To construct the validation of the present system and method, the system visually compares the result of the hybrid segmentation algorithm to a ground true result obtained on fixed images. In order to get a representative data set, the system can extract an image every two second from the video of the patient, and 6 videos of the ADAPT series with the first visit of 6 patients. The 6 patients were diverse with three women, three men, one African American/Black, one Asian, one Hispanic, three white.

In one embodiment, for testing, the system extracts one image every 2 seconds of the video clip for Exercise 1 assessing ptosis and the two video clips corresponding to Exercise 2 assessing eyes misalignment. It does the same with the patient video who is registered with the Inteleclinic system equipped with a high-definition camera. Each Exercise lasts about one minute, so the system gets a total of about 540 images from the ADAPT series and 90 from the Inteleclinic one. The validation of the image segmentation is done for each eye which doubles the amount of work.

For Exercise 1, the system checks 3 landmarks positions: the points on the upper lid, iris bottom and lower lid situated on the vertical line that cross the center of the ROI. For Exercise 2, the system looks for the position of the iris boundary that is opposite to the direction that the patient looks at: if the patient looks on his/her left the system checks on the position of the iris boundary point that is the further on the right.

To facilitate the verification, the code automatically generates these images with an overlay a grid of spatial steps 2 pixels. This rule is plugged vertically for Exercise 1 and horizontally for Exercise 2.

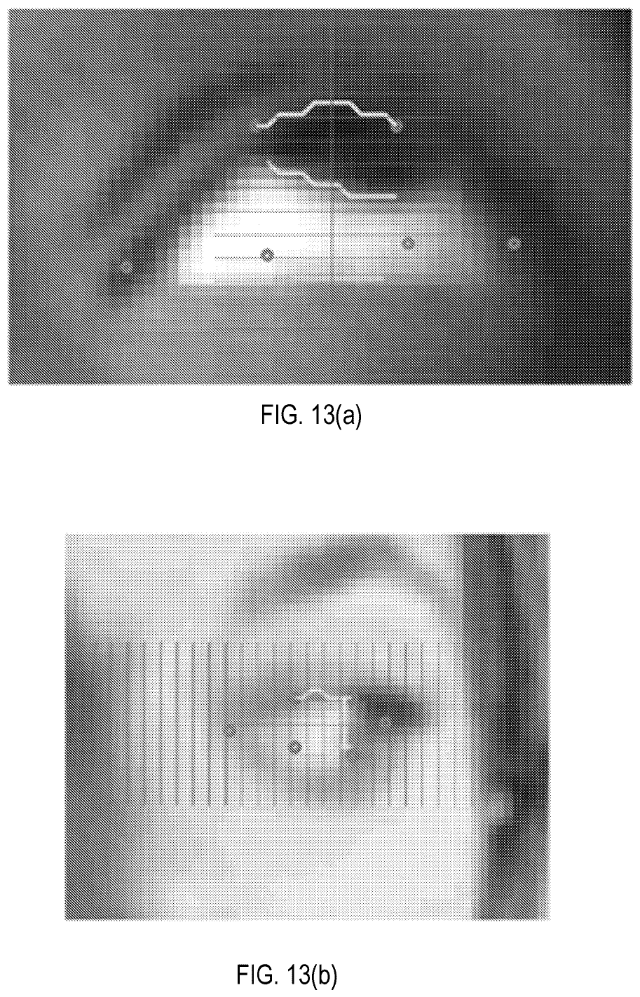

We consider that the segmentation is correct, to assess ptosis and ocular misalignment, when the localization of the landmarks is correct within 2 pixels. It is often difficult to judge visually on the results, as shown in the image zoomed of FIGS. 13(a), 13(b). The system uses two independent visual verification of reviewers to validate the results. Two pixels error means that the interface is localized within 2 pixels. For a curve projected on a pixelized grid, this is about the optimum accuracy you can state on the interface location using pixels.

Not all images are resolved by the hybrid algorithm. However, the system keeps enough time frames in the video to reconstruct the dynamic of ptosis and possible ocular misalignment. First, the system eliminates from the data set of images, all the images in which the Deep Learning library fails to localize correctly the eyes. This can be easily detected in a video, since the library operates on each frame individually and may jump from one position to a completely different one while the patient stays still. For example, for one of the patients, the deep learning algorithms confuse randomly the two nostrils with the eyes.

The Adapt video series has low resolution, especially when the displays are side by side of the patient and the medical doctor, and may suffer from poor contrast or image focus or condition of lightning so it is not particularly surprising that the system can keep on average only 74% of the data set for further processing with the hybrid algorithm.

The system and algorithm also cannot find precisely the landmark being looked for, when the deep learning library gives an ROI that is significantly off the target. The bias on the deep learning algorithm is particularly significant during Exercise 1, where the eyes is wide open and the sclera area all decentered below the iris. The lower points of the polygon that mark the ROI are often far inside the white sclera above the lower lid. The end points of the hexagon in the horizontal direction may get misaligned with the iris too far off the rectangular area of local search that the system is to identify.

We eliminate automatically 44% of the images of the video clips of the ADAPT series, and 10% of the Inteleclinic series for Exercise 1. The Inteleclinic result was acquired in better lightning condition with also a higher resolution than the ADAPT series.

For Exercise 1 with the ADAPT series, the system obtains a success rate of 73% for the lower lid, 89% for the bottom of the iris and 78% for the upper lid. For Exercise 1 and the Inteleclinic series of images the system obtains a success rate respectively of 77%, 100%, and 77%

For Exercise 2, the quality of the acquisition is somehow better: 18% of the image ROIs for the ADAPT series but about the same, i.e., 13% for the Inteleclinic series.

Globally the localization of the iris boundary used to check ocular misalignment is better with a success rate of 95%. The eyes are less open than in Exercise 1 and closer to “normal” shape: the upper lid, respectively lower lid landmarks are obtained with a success rate respectively of 73% and 86%.

### Ptosis and Diplopia Assessment

As illustrated in FIG. 5(a), the system can determine from the polygon obtained by the deep learning algorithm, a first approximation of ptosis level by computing the area of the eyes that is exposed to the view as well as the vertical dimension of the eyes. As a byproduct of this metric, the system may identify blinking, see FIG. 10. The left and right eyes blinking at the same time is expected. Surprisingly not every patient diagnosed with MG are blinking during the Exercise, though the clinical significance of this remains to be studied. This computing can occur, for example, as part of or following the density check, step 326. Computing blinking requires that there are very small holes at best, since blinking takes a fraction of a second. On the other hands blinking works just with the deep learning algorithm of the model of eyes and may not need accurate computer vision corrections, and can detect the time when eyes are closed.

The time dependent measure of diplopia or ptosis obtained by the present algorithm contains noise. The system 100 can improve the accuracy of the measures by ignoring, step 330, the eyes with identified detection outliers (and artifacts) provided that the time gaps corresponding to these outliers are small, step 328. To recover the signal without losing accuracy, the system can use any suitable process, such as a high order filtering technique, step 330, to analyze thermal imagery signal [13].

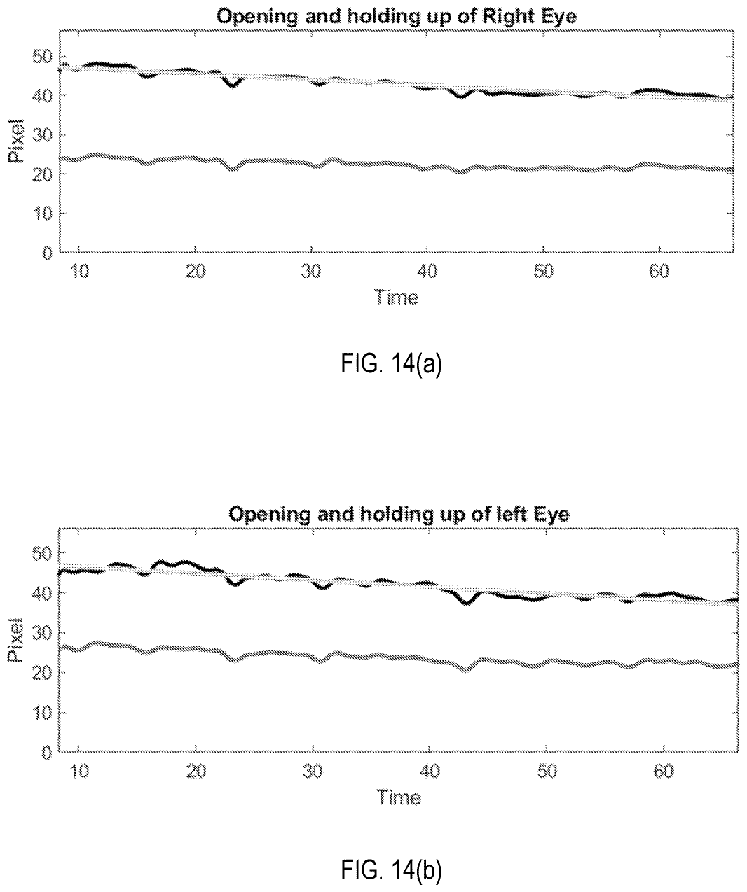

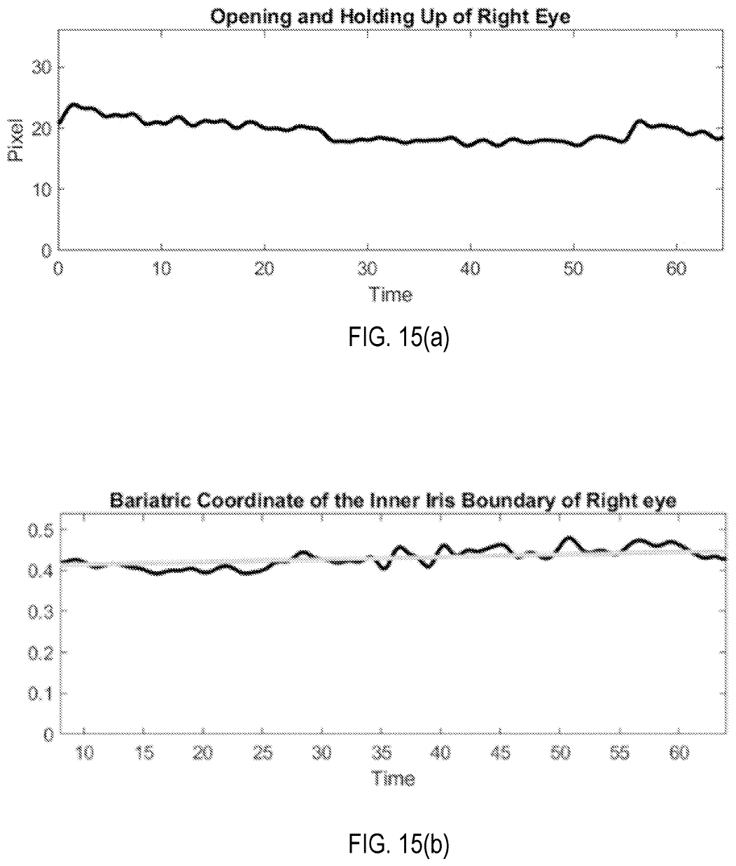

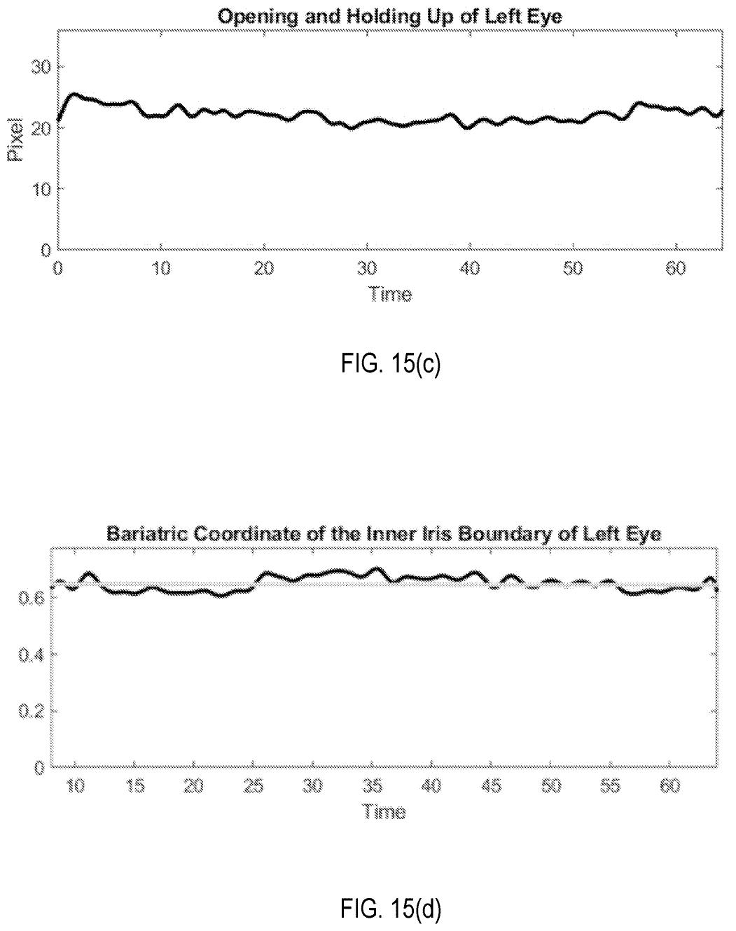

Step 332 corresponds to the numbers that come from the graph of FIGS. 14(a), 14(b), 15(b), 15(d). For example, the measure of the slope of the green lines that have been obtained by least square fitting.

At step 334, the reports of FIG. 18 are generated. Step 336 means no report, and the data acquisition has to be done again. This would be typic if the patient moves too much or is too far from the camera, or light conditions are a disaster. As shown, the system generates a result for a number of patient characteristics, including Distance Upper Lid—Pupil, Alignment Eyes, Arm Fatigue, Sit to Stand, Speech Analysis, and Cheek Puff. The Distance Upper Lid—Pupil is a measurement of the distance 707 (FIG. 5(c)) between the upper lid and the center of the pupil. That distance is more accurately measured following the computer vision analysis, steps 308, 310. The Alignment Eyes indicates the misalignment (deviation of alignment) between the left and right eyes. As noted above, one measure of misalignment is the difference between α_{left} and α_{right} may change with time and corresponds to the misalignment of both eyes.

FIG. 18 further illustrates that the current disclosure can be applied to other patient reports, such as Arm Fatigue, Sit to Stand, Cheek Puff, whereby AI and computer vision are combined to obtain both the robustness of AI and the accuracy of computer vision. Speech Analysis does not use computer vision, if only speech is involved, but speech based on mouth motion can be analyzed by the present system with AI and computer vision. Accordingly, though the disclosure is directed to eye feature identification and tracking, the system is generic and can be applied to many situations beyond eye tracking. For example, it can be applied to reconstruct any specific anatomic marker accurately in a video or image, such as arm, check and overall body structure and/or movement (distance, speed, rate, etc.). In addition, although the disclosure is directed to MG, the system has applications beyond MG, including for example multiple sclerosis, and Parkinson, where for example, the system assesses hand motion, walking balance, tremoring.

Static is a measure independent of time, such as for example, the eye opening at the start or the end of the exercise. Dynamic means the time dependent variation of eye opening. In the graphs, the y coordinate of the graph are in pixels, and the x coordinate is time in seconds. The outer arch shape is the scale or gauge against which the patient's results can be easily measured. In the gauge, the first zone (the leftmost) is good, the second zone is OK, the third zone is bad, and the last zone (the rightmost) is very bad. The inner curve and the numerical value (e.g., 0.8 for Alignment Eyes is in the first zone, whereas 2.4 for Speech Analysis is in the third zone) is the patient's score/result, which is easily viewed by the practitioner by aligning the patient's score to the outer scale. The patient would want all indicators to the left. The trend is the comparison between this report and the previous one. Based on the results

The Inteleclinic data set is working well as shown in FIGS. 14(a), 14(b). The upper straight line shows a least square approximation of the distance between the lower lid and upper lid of the patient. The lower curve shows the distance between the lower point of the iris and the lower lid below. This second curve is used to check that the patient does the Exercise correctly.

We observe a 15% decay in eye opening that is very difficult to appreciate visually on the video clip, or during the medical doctor examination. This low shift of the upper lid is slow and almost unnoticeable during a 60 second observation. This is the least square lines of FIGS. 14 and 15, i.e., a standard linear square fitting of each curve.

During Exercise 2, the system obtains no eye misalignment for the same patient, but the eye opening is about half of its value during the first ptosis Exercise and the eye opening does not stay perfectly constant. On the Inteleclinic video, the eye gaze direction to the left and to the right is so extreme that one of the pupils might be covered in part by the skin at the corner of the eyes, which may question the ability of the patient to experience diplopia in that situation.

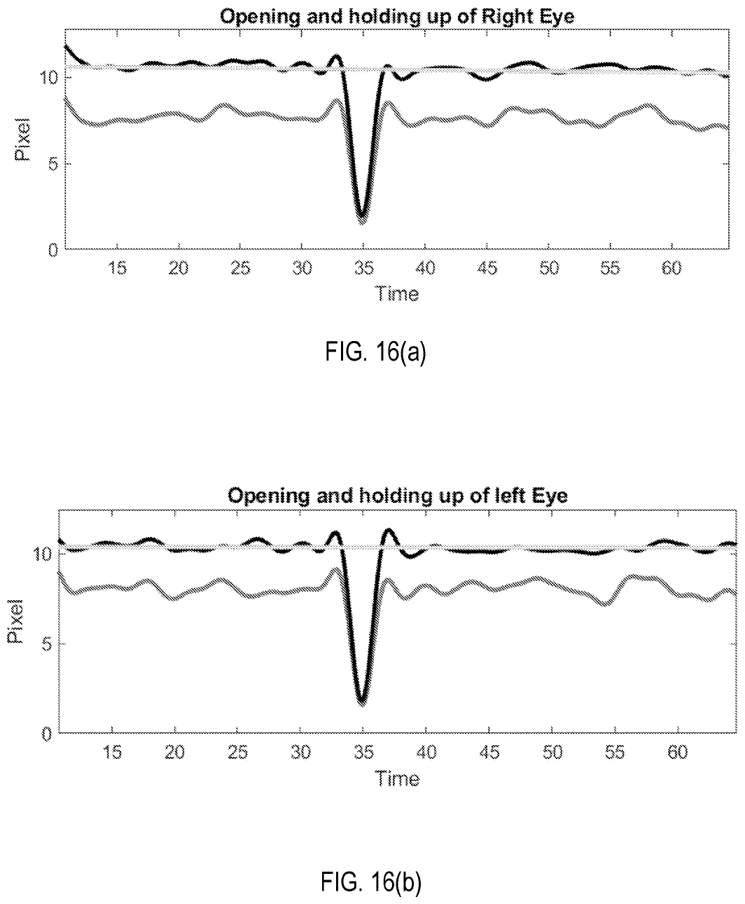

The results of ptosis and diplopia for the ADAPT video are less effective but still allow an assessment of ptosis and diplopia, though with less accuracy. FIGS. 16(a), 16(b) show a representative example of the limit of the method, when the gap of information between two time points cannot be recovered. It should be appreciated that the eye opening was of the order of 10 pixels as opposed to about 45 in the inteleclinic data set. The patient was not close enough to the camera during the Exercise which makes the resolution even worst. However, the system could check a posteriori that the gap found by the algorithm does correspond to a short period of time when the patient loses their upper eye gaze position and relax to look straight.

FIGS. 15(a)-(d) show the evolution of the barycentric coordinates of each eye during the second Exercise. A normal subject is making a convergence movement, which leads to rotation of each eye towards the midline. If there is no eyes misalignment building up in the exercise, the least square line should be horizontal, as shown in FIG. 15(d), which means that this patient is “normal”

### DISCUSSION AND CONCLUSION

Due to the precautions caused by the COVID-19 pandemic, there has been a rapid increase in the utilization of TM in patient care and clinical trials. The move to video evaluations offers the opportunity to objectify and quantify the physical examination, which presently relies on the subjective assessment of an examiner with varied levels of experience and often limited time to perform a thorough examination. Physicians still remain reticent to incorporate TM into their clinical habits, in particular in areas that require a physical examination (neuromuscular diseases, movement disorders) compared to areas that are primarily symptom-focused (headache). Telemedicine, on the other hand, has numerous features to provide an enhanced assessment of muscle weaknesses, deeper patient monitoring and education, reduced burden and cost of in-person clinic visits, and increased patient access to care. The potential for clinical trials to establish rigorous, reproducible examinations at home provides similar benefits for research subjects.

MG is an autoimmune neuromuscular disease with significant morbidity that serves as a reference for other targeted therapies. Outcome measures are established for MG trials, but these are considered suboptimal [33]. The MG core examination, in particular ocular MG, has been standardized and is well defined [5]. Because of the high frequency of consultation for MG patient, teleconsultation is now commonly used in the US. However, the grading of ptosis and diplopia relies on a repetitive and tedious examination that the medical doctor must perform. The dynamic component of upper eyelid dropping is overlooked during the examination. Diagnosis of diplopia in these telehealth sessions rely on patient subjective feedback. Overall, the physical examination relies heavily on qualitative experienced judgment rather than on unbiased rigorous quantitative metrics.

One goal of the system and method of the present disclosure is to move from 2D teleconsultation and its limitation to a multi-dimension consultation. The system presented in this paper addresses that need by introducing modern image processing technique that are quick and robust to recover quantitative metrics that should be independent of the examiner. The diagnosis and treatment decisions remain the responsibility of the medical doctor who has the medical knowledge and not the algorithm output.

One of the difficulties of standard telehealth sessions is the poor quality of video. The resolution may be severely limited by the bandwidth of the network at the patient location. In the trial, the quality of the video was certainly enough to let the medical doctor assess ptosis and diplopia as specified above, but not great for image processing especially because the videos were recorded on the doctor side rather than recording the raw video footages on the patient side. Light conditions and positioning of the patient in front of the camera was often poorly controlled when patients are at home with their personal computer or tablet. It is of crucial importance to privilege numerical algorithm and image processing that are robust and transparent on the level of accuracy they provide. Eye tracking in particular is very sensitive to patient motion, poor resolution of the image and eventually eyelid dropping or gaze directed on the side.

As the Exercise output is digitalized to assess ptosis, the system has to define rigorously the metric. The system can look at instantaneous measurement as well as time dependent one: from the dynamic perspective to discriminate patient who shows steady upper eyelid drop from those who start well and get progressive eyelid drop. The system can also separate: global measurement related to the overall eye opening, from measurement that compute the distance from the pupil to the upper lid. This last metric is clinically significant for the patient when the drop is such that it impairs vision. A decision on how these metrics should be classify as ptosis grade remains to be done on accordance with medical doctor.

Similarly, diplopia can be measured by the “misalignment” of the left and right pupil during Exercise 2. Vision indeed is a two stages process where the brain can compensate for some of the misalignment and cancel the impairment.

Both measurement of ptosis and diplopia are quite sensitive to the resolution of the video. In Zoom recorded telehealth session, the distance from the pupil to the upper lid is of the order of 10 pixels. A 2-pixel error on the landmark positions may still provide a relative error of about 20% on the ptosis metric. The deep learning algorithm introduce even larger errors on the landmark points of the ROI polygon. However, with a HD camera, and the processing being done on raw footage rather than on streamed recorded footage, this relative error gets divided by two.

The system approach can also be used to provide recommendations on how to improve the MG ocular exam. For example, to ensure the reproducibility and quality of the result, the algorithm can provide feedback in real-time to the medical doctor on how many pixels are available to track the eyes and therefore give direction to the patient to position closer and better with respect to the camera on her/his ends. Similarly Exercise 2 may benefit from reduced extreme eccentric gaze that the one seen in video, in a way that the iris boundary does not get covered by the skin. This would allow for a more realistic situation to assess double vision properly.

Development of a model of the eye geometry with its iris and pupil geometric marker that extend the model of FIG. 5(a) in greater detail including upper lid drop can also be provided. Applying deep learning technology to this model would be quite feasible, though would require hundreds of patient and video with correct annotation to train the algorithm [19]. Further, deep learning technology may have spectacular robustness that are shown in annotated videos but may not guaranty accuracy. A high-performance telehealth platform [34] can also be provided that can be conveniently distributed at multiple medical facilities to build the large, annotated quality data set to advance understating of MG.

It is noted that a number of components and operations are shown and described, for example with respect to FIGS. 1-4, 6. However, not all of the components need to be provided, such as for example, lighting system 114, laser pointer 550, beeper 370, buttons 410, 420, thermal camera 250, sensors 540, 580. And, not all of the operations need to be provided, such as determining state variables 810, digital twins 800, physical, emotive, cognitive and/or social variables 820, 840, 860, 880, or the operations of FIGS. 6(a)-(c). But rather a more generic telehealth or video conferencing system can be provided without those features. Moreover, the present system and process can be implemented on a stand-alone system at the practitioner's office (FIG. 2), such as just prior to examination by a physician, and not over a video conferencing or telehealth system. Or, the patient can capture video at the patient system 200 and send it (e.g., email or upload to a website) to the practitioner or remote site. In addition, the analysis can occur at the patient system 200 or at the practitioner system 120. Still further, the deep learning and computer vision analysis portion fo the system 100 can be implemented by itself, and not in a telehealth system, for example on a cell phone, or any smart camera, to improve the outcome where any eye tracking device can be useful.

Clinical trials require close monitoring of subjects at multiple weekly and monthly check-in appointments. This time requirement disadvantages subjects who cannot leave family or job obligations to participate or are too sick to travel to any medical center, many of which are located large distances from their homes. This limitation compromises clinical trial recruitment and the diversity of subjects. Clinical trials are also expensive, and reducing costs is a primary goal for these companies. The method for eye tracking offers the potential to lower clinical research costs through the following methods: (i) Increasing enrollment through increased patient access; (ii) Reducing the workload on staff through increased automated tasks; (iii) Diversifying subject enrollment which increases the validity of the studies and leads to better scientific discoveries; and (iv) Improving data collection by providing unbiased core exam data through AI, computer vision.

The following references are hereby incorporated by reference.
[1] M. Giannotta, C. Petrelli, et A. Pini, «Telemedicine applied to neuromuscular disorders: focus on the COVID-19 pandemic era», p. 7.
[2] E. Spina et al., «How to manage with telemedicine people with neuromuscular diseases?», Neurol. Sci., vol. 42, no 9, p. 3553-3559, sept. 2021, doi: 10.1007/s10072-021-05396-8.
[3] S. Hooshmand, J. Cho, S. Singh, et R. Govindarajan, «Satisfaction of Telehealth in Patients With Established Neuromuscular Disorders», Front. Neurol., vol. 12, p. 667813, mai 2021, doi: 10.3389/fneur.2021.667813.
[4] D. Ricciardi et al., «Myasthenia gravis and telemedicine: a lesson from COVID-19 pandemic», Neurol. Sci., vol. 42, no 12, p. 4889-4892, dec. 2021, doi: 10.1007/s10072-021-05566-8.
[5] A. Guidon, S. Muppidi, R. J. Nowak, J. T. Guptill, M. K. Hehir, K. Ruzhansky L. B., Burton, D. Post, G. Cutter, R. Conwit, N. I. Mejia, H. J. Kaminski, J. F. Jr. Howard, Telemedicine visits in myasthenia gravis: Expert guidance and the myasthenia gravis core exam (MG-CE) Muscle Nerve 2021; 64:270-76.
[6] Jan Lykke Scheel Thomsen and Henning Andersen, Outcome Measures in Clinical Trials of Patients With Myasthenia Gravis, Front. Neurol., 23 Dec. 2020, Sec. Neuromuscular Disorders and Peripheral Neuropathies, https://doi.org/10.3389/fneur.2020.596382
[7] M. Al-Haida, M. Benatar and H. J. Kaminski, Ocular Myasthenia, Neurologic Clinics Volume 36, Issue 2, May 2018, Pages 241-251.
[8] G. Liu, Y. Wei, Y. Xie, J. Li, L. Qiao, et J.-J. Yang, «A computer-aided system for ocular myasthenia gravis diagnosis», Tsinghua Sci. Technol., vol. 26, no 5, p. 749-758, oct. 2021, doi: 10.26599/TST.2021.9010025.
[9] An Tang et Al, Health Policy and Practice/Sante: politique et pratique medicale, Canadian Association of Radiologists White Paper on Artificial Intelligence in Radiology, canadian Association of Radiologist Journal 69, 120-135, 2018.
[10] Leigh, R. John, and David S. Zee, The Neurology of Eye Movements, 5 edn, Contemporary Neurology Series (New York, 2015; online edn, Oxford Academic, 1 Jun. 2015), https://doi.org/10.1093/med/9780199969289.001.0001, accessed 12 Aug. 2022.
[11] M.1 D. Crutcher, R. Calhoun-Haney, C. M. Manzanares, J. J. Lah, Al. I. Levey, P. S. M. Zola, Eye Tracking During a Visual Paired Comparison Task as a Predictor of Early Dementia, American Journal of Alzheimer's Disease & Other Dementias, Vol 24 Number 3, June/July 2009 258-266
[12] J. Thomas Hutton, J. A. Nagel, Ruth B. Loewenson, Eye tracking dysfunction in Alzheimer-type dementia, Neurology Jan 1984, 34 (1) 99; DOI: 10.1212/WNL.34.1.99
[13] M Garbey, N Sun, A Merla, I Pavlidis, Contact-free measurement of cardiac pulse based on the analysis of thermal imagery, IEEE transactions on Biomedical Engineering 54 (8), 1418-1426.
[14] T. M. Burns, M. Conaway, et D. B. Sanders, «The MG Composite: A valid and reliable outcome measure for myasthenia gravis», Neurology, vol. 74, no 18, p. 1434-1440, 2010, doi: 10.1212/WNL.0b013e3181dc1b1e.
[15] F. Rynkiewicz, M. Daszuta, et P. Napieralski, Pupil Detection Methods for Eye Tracking, Journal of Applied Computer Science, Vol. 26 No. 2 (2018), pp. 201-21.
[16] Dan Witzner Hansen, Qiang Ji, “In the eye of the beholder: a survey of models for eyes and gaze,” IEEE Transactions on Pattern Analysis and Machine Intelligence. Vol. 32, No. 3, pp. 478-500, 2010.
[17] Hari Singh and Jaswinder Singh, Human Eye Tracking and Related Issues: A Review, International Journal of Scientific and Research Publications, Volume 2, Issue 9, September 2012 1 ISSN 2250-3153.
[18] W. Khan, A. Hussain, K. Kuru, et H. Al-askar, «Pupil Localisation and Eye Centre Estimation Using Machine Learning and Computer Vision», Sensors, vol. 20, no 13, p. 3785, juill. 2020, doi: 10.3390/s20133785.
[19] Zhao, Lei; Wang, Zengcai; Zhang, Guoxin; Qi, Yazhou; Wang, Xiaojin (15 Nov. 2017). “Eye state recognition based on deep integrated neural network and transfer learning”. Multimedia Tools and Applications. 77 (15): 19415-19438. doi:10.1007/s11042-017-5380-8.
[20] Bartosz Kunka and Bozena Kostek, Non-intrusive infrared-free eye tracking method, Conference: Signal Processing Algorithms, Architectures, Arrangements, and Applications Conference Proceedings (SPA), 2009, IEEE Xplore.
[21] A. A. Ghali, S. Jamel, K. M. Mohamad, N. A. Yakub, et M. M. Deris, «A Review of Iris Recogntion Algorithms», p. 4.
[22] K. Toennies, F. Behrens, M. Aurnhammer. Feasibility of hough-transform-based iris localization for real-timeapplication. In 16th International Conference on Pattern Recognition, 2002. Proceedings, vol. 2, 1053-1056, 2002.
[23] D. B. B. Liang, L. K. Houi. Non-intrusive eye gaze direction tracking using color segmentation and Hough transform. International Symposium on Communications and Information Technologies, 602-607, 2007.
[24] Prateek Verma, Maheedhar Dubey, Praveen Verma, Somak Basu, Daughman's Algorithm Method for Iris Recognition—a Biometric Approach, International Journal of Emerging Technology and Advanced Engineering, Website: www.ijetae.com (ISSN 2250-2459, Volume 2, Issue 6, June 2012)
[25] V. Jain et E. Learned-Miller, «FDDB: A Benchmark for Face Detection in Unconstrained Settings», p. 11.
[26] A. T. Kabakus, «An Experimental Performance Comparison of Widely Used Face Detection Tools», ADCAIJ Adv. Distrib. Comput. Artif. Intell. J., vol. 8, no 3, p. 5-12, sept. 2019, doi: 10.14201/ADCAIJ201983512.
[27] OpenCV Haar Cascade Eye detector. [En ligne]. Disponible sur: https://github.com/opencv/opencv/blob/master/data/haarcascades/haarcascade_eye.xml
[28] M. H. An, S. C. You, R. W. Park, et S. Lee, «Using an Extended Technology Acceptance Model to Understand the Factors Influencing Telehealth Utilization After Flattening the COVID-19 Curve in South Korea: Cross-sectional Survey Study», JMIR Med. Inform., vol. 9, no 1, p. e25435, janv. 2021, doi: 10.2196/25435.
[29] B. Johnston et P. de Chazal, «A review of image-based automatic facial landmark identification techniques», EURASIP J. Image Video Process., vol. 2018, no 1, p. 86, d6c. 2018, doi: 10.1186/s13640-018-0324-4.
[30] X. Cao, Y. Wei, F. Wen, et J. Sun, «Face Alignment by Explicit Shape Regression», Int. J. Comput. Vis., vol. 107, no 2, p. 177-190, avr. 2014, doi: 10.1007/s11263-013-0667-3.
[31] V. Kazemi et J. Sullivan, «One millisecond face alignment with an ensemble of regression trees», in 2014 IEEE Conference on Computer Vision and Pattern Recognition, Columbus, OH, juin 2014, p. 1867-1874. doi: 10.1109/CVPR.2014.241.
[32] C. Sagonas, E. Antonakos, G. Tzimiropoulos, S. Zafeiriou, et M. Pantic, «300 Faces In-The-Wild Challenge: database and results», Image Vis. Comput., vol. 47, p. 3-18, mars 2016, doi: 10.1016/j.imavis.2016.01.002.
[33] Reports and Data. (2022, Jan. 3). Myasthenia Gravis Market Size, Share, Industry Analysis By Treatment, By End-Use and Forecast to 2028. Retrieved from BioSpace: https://www.biospace.com/article/myasthenia-gravis-market-size-share-industry-analysis-by-treatment-by-end-use-and-forecast-to-2028/
[34] A smart Cyber Infrastructure to enhance usability and quality of telehealth consultation, M. Garbey, G. Joerger, provisional 63305420 filed by GWU, January 2022.
[35] M Garbey, N Sun, A Merla, I Pavlidis, Contact-free measurement of cardiac pulse based on the analysis of thermal imagery, IEEE transactions on Biomedical Engineering 54 (8), 1418-1426.

It is noted that the drawings may illustrate, and the description and claims may use geometric or relational terms, such as right, left, upper, lower, side (i.e., area or region), length, width, top, bottom, rectangular, etc. These terms are not intended to limit the disclosure and, in general, are used for convenience to facilitate the description based on the examples shown in the figures. In addition, the geometric or relational terms may not be exact.

While certain embodiments have been described above, those skilled in the art who have reviewed the present disclosure will readily appreciate that other embodiments can be realized within the scope of the invention. Accordingly, the present invention should be construed as limited only by any appended claims.

1. An image detection system, comprising:
a processing device configured to receive image data of a patient's face, apply deep learning to identify an initial region of interest and initial landmark points corresponding to the patient's eyes, apply computer vision to refine the initial landmark points, and determine ptosis and/or diplopia based on the refined landmark points.

2. The image detection system of claim 1, said processing device configured to generate a bounding box at the initial landmark points corresponding to the patient's eyes, identify a lower eyelid interface between the patient's sclera and the patient's skin corresponding to the lower lid, identify a lower iris interface between the patient's iris and the patient's sclera.

3. The image detection system of claim 1, wherein said image detection system is integrated in a telehealth system or a video conferencing system.

4. (canceled)

5. The image detection system of claim 1, said processing device for eye segmentation and eye tracking.

6. The image detection system of claim 1, wherein the computer vision is applied to the patient's iris and pupil with 2-pixel accuracy on average.

7. The image detection system of claim 1, wherein ptosis and diplopia are used to detect a neurological disease in the patient.

8. The image detection system of claim 7, wherein the neurological disease is Myasthenia Gravis.

9. The image detection system of claim 1, wherein the image data is a fixed image or a video.

10. (canceled)

11. An image detection system, comprising:
a processing device configured to receive annotated image data of a patient's face annotated with an initial region of interest and initial landmark points corresponding to the patient's eyes, apply computer vision to refine the initial landmark points, and determine ptosis and/or diplopia based on the refined landmark points.

12. The system of claim 11, wherein the annotated image data is determined from deep learning of image data.

13. The image detection system of claim 11, said processing device configured to generate a bounding box at the initial landmark points corresponding to the patient's eyes, identify a lower eyelid interface between the patient's sclera and the patient's skin corresponding to the lower lid, identify a lower iris interface between the patient's iris and the patient's sclera.

14. The image detection system of claim 11, wherein said image detection system is integrated in a telehealth system or a video conferencing system.

15. (canceled)

16. The image detection system of claim 11, said processing device for eye segmentation and eye tracking.

17. The image detection system of claim 11, wherein the computer vision is applied to the patient's iris and pupil with 2-pixel accuracy on average.

18. The image detection system of claim 11, wherein ptosis and diplopia are used to detect a neurological disease in the patient.

19. The image detection system of claim 18, wherein the neurological disease is Myasthenia Gravis.

20. The image detection system of claim 11, wherein the image data is a fixed image or a video.

21. (canceled)

22. An image detection system, comprising:
a processing device configured to receive image data of a patient's body, apply deep learning to identify an initial region of interest and initial landmark points, apply computer vision to refine the initial landmark points, and determine a patient disorder based on the refined landmark points.

23. The system of claim 22, wherein the patient disorder comprises Myasthenia Gravis, ptosis, diplopia multiple sclerosis or Parkinson.

24. The system of claim 22, wherein the landmark points comprise a patient's eye, hand, body, arm, or leg.

25. The system of claim 22, said processing device further configured to determine eye fatigue, hand motion, sit to stand, speech analysis based on mouth movement, cheek puff, walking balance, tremoring, and/or body interfaces based on the refined landmark points.

26. The system of claim 22,
wherein the image data comprises annotated image data of a patient's body annotated with the initial region of interest and the initial landmark points.

27. (canceled)

28. (canceled)

29. (canceled)
